(19) United States
(12) Patent Application Publication
(10) Pub. No.: US 2018/0153850 A1
(43) Pub. Date: Jun. 7, 2018

(54) COMPOSITIONS AND METHODS FOR TREATMENT OF CANCER

(71) Applicant: The Regents of the University of California, Oakland, CA (US)

(72) Inventors: Allen C. Gao, Davis, CA (US); Yezi Zhu, Davis, CA (US)

(73) Assignee: The Regents of the University of California, Oakland, CA (US)

(21) Appl. No.: 15/796,211

(22) Filed: Oct. 27, 2017

Related U.S. Application Data
(63) Continuation of application No. PCT/US2016/030126, filed on Apr. 29, 2016.
(60) Provisional application No. 62/156,033, filed on May 1, 2015.

(51) Int. Cl. A61K 31/337; A61K 31/4166; A61P 35/00; A61K 31/277

(52) U.S. Cl. CPC A61K 31/337; A61K 31/277; A61P 35/00; A61K 31/4166

(57) ABSTRACT

Methods and compositions are provided for treatment of cancer, including prostate cancer. In one aspect, the present invention provides a composition comprising a taxane and a drug that reduces efflux of the taxane from a cell. The drug that reduces efflux of the taxane can be an inhibitor of ABCB1 efflux activity. In some cases, the inhibitor of ABCB1 efflux activity is an inhibitor of ABCB1 ATPase activity selected from the group consisting of enzalutamide and bicalutamide.

### CROSS-REFERENCES TO RELATED APPLICATIONS

This application is a continuation of International Patent Application No. PCT/US2016/030126, filed Apr. 29, 2016, which claims priority to U.S. Provisional Application No. 62/156,033, filed May 1, 2015, the disclosures of which are hereby incorporated by reference in their entirety for all purposes.

### STATEMENT AS TO RIGHTS TO INVENTIONS MADE UNDER FEDERALLY SPONSORED RESEARCH AND DEVELOPMENT

This invention was made with Government support under Grant No. CA179970, awarded by the National Cancer Institute. The government has certain rights in this invention.

### BACKGROUND OF THE INVENTION

Cancer is a leading cause of death in the United States. Prostate cancer is the most frequently diagnosed cancer and second most common cause of cancer-related death among men in the United States. Prostate cancer cells are dependent on androgen receptor (AR) signaling for growth and survival (1). Therefore, most prostate cancer patients initially respond to androgen deprivation therapy (ADT). However, the majority will eventually develop castration-resistant prostate cancer (CRPC) (2, 3). The taxane docetaxel, a standard first line treatment for CRPC, inhibits CRPC growth by binding β-tubulin and inhibiting mitotic cell division, leading to apoptotic cell death (4, 5). However, only approximately 50% of patients respond to docetaxel and even those who initially benefit from the treatment eventually develop resistance to the drug (6, 7).

### BRIEF SUMMARY OF THE INVENTION

In one aspect, the present invention provides a composition comprising a taxane and a drug that reduces efflux of the taxane from a cell. In some embodiments, the drug that reduces efflux of the taxane comprises an inhibitor of ABCB1 efflux activity. In some embodiments, the inhibitor of ABCB1 efflux activity comprises an inhibitor of ABCB1 ATPase activity. In some embodiments, the drug that reduces efflux of the taxane comprises an anti-androgen drug. In some cases, the anti-androgen drug is selected from the group consisting of enzalutamide, bicalutamide, and combinations thereof. In some cases, the anti-androgen drug is an inhibitor of ABCB1 efflux activity. In some cases, the anti-androgen drug is an inhibitor of ABCB1 ATPase activity. In some cases, the taxane is selected from the group consisting of paclitaxel, docetaxel, cabazitaxel, taxol, hongdoushan A, hongdoushan B, hongdoushan C, baccatin I, baccatin II, a 10-deacetylbaccatin, and a combination thereof.

In some embodiments, the composition comprises docetaxel and enzalutamide or bicalutamide. In some cases, the composition comprises docetaxel and enzalutamide. In some cases, the composition comprises docetaxel and bicalutamide. In some cases, the composition is an effective inhibitor of cancer cell proliferation. In some cases, the composition is a more effective inhibitor of cancer cell proliferation as compared to a taxane alone, or as compared to an anti-androgen drug alone. In some cases, the composition exhibits synergistic effectiveness in reducing cancer cell proliferation as compared to a taxane alone or an anti-androgen drug alone. In some cases, the drug that reduces efflux of the taxane from the cell in the composition increases the plasma concentration of the taxane in a subject as compared to the plasma concentration of the taxane in a subject having received the taxane alone.

In some cases, the composition is an effective inhibitor of proliferation of a cancer cell selected from the group consisting of a prostate cancer cell, a metastatic prostate cancer cell, a castrate-resistant prostate cancer cell, a castration recurrent prostate cancer cell, an androgen insensitive prostate cancer cell, a hormone-resistant prostate cancer cell, and a metastatic castrate-resistant prostate cancer cell. In some cases, the composition is an effective inhibitor of proliferation of a taxane-resistant cancer cell. In some cases, the composition is an effective inhibitor of proliferation of a docetaxel-resistant cancer cell. In some cases, the composition is an effective inhibitor of proliferation of a cancer cell (e.g., taxane-resistant cancer cell) that exhibits increased expression or activity of ABCB1. In some cases, the composition is a more effective inhibitor of proliferation of a cancer cell (e.g., taxane-resistant cancer cell) that exhibits increased expression or activity of ABCB1 as compared to a taxane alone, or as compared to an anti-androgen drug alone. In some cases, the expression or activity of ABCB1 in the cancer cell is increased relative to a cancer cell prior to a start of treatment (e.g., taxane treatment), prior to onset of resistance to a treatment, or in comparison to an in vitro control cell line (e.g., LNCaP, DU145, CWR22RV1 and C4-2B etc.).

In a second aspect, the present invention provides a method for treating cancer comprising administering an effective amount of a composition of any one of the foregoing compositions, or a combination thereof. In some embodiments, the cancer is prostate cancer. In some embodiments, the prostate cancer is selected from the group consisting of metastatic prostate cancer, castrate-resistant prostate cancer, castration recurrent prostate cancer, androgen insensitive prostate cancer, hormone-resistant prostate cancer, and metastatic castrate-resistant prostate cancer. In some cases, the prostate cancer is taxane-resistant prostate cancer. In some cases, the taxane-resistant prostate cancer is docetaxel-resistant prostate cancer.

In a third aspect, the present invention provides a method for reducing or reversing resistance of a taxane-resistant cell to a taxane, or re-sensitizing a taxane-resistant cell to a taxane, the method comprising contacting the cell with an effective amount of an anti-androgen drug selected from the group consisting of enzalutamide, bicalutamide, or a combination thereof. In some embodiments, the method includes contacting the cell with an effective amount of any one of the foregoing compositions, or combinations thereof. In some embodiments, the cell is docetaxel-resistant, and the method comprises reducing or reversing docetaxel resistance of the docetaxel-resistant cell or re-sensitizing the docetaxel-resistant cell to docetaxel. In some embodiments, the cell is a cancer cell. In some embodiments, the cancer cell is a prostate cancer cell. In some cases, the prostate cancer cell is selected from the group consisting of metastatic prostate cancer, castrate-resistant prostate cancer, castration recurrent prostate cancer, androgen insensitive prostate cancer, hormone-resistant prostate cancer, and metastatic castrate-resistant prostate cancer. In some cases, the prostate cancer cell is docetaxel-resistant, and the method comprises reducing or reversing docetaxel resistance of the docetaxel-resistant prostate cancer cell or re-sensitizing the docetaxel-resistant prostate cancer cell to docetaxel.

In a fourth aspect, the present invention provides a method for treating cancer in a subject in need thereof, the method comprising: simultaneously or sequentially administering to the subject a taxane, and an inhibitor of taxane efflux, wherein the inhibitor of taxane efflux comprises an anti-androgen drug. In some cases, the anti-androgen drug comprises bicalutamide or enzalutamide, a derivative thereof, or a salt thereof. In some cases, the method further comprises simultaneously or sequentially administering to the subject a second inhibitor of taxane efflux. In some cases, the method comprises simultaneously or sequentially administering the taxane, bicalutamide and enzalutamide. In some cases, the cancer is a taxane-resistant cancer.

In a fifth aspect, the present invention provides a method for inhibiting ABCB1 efflux in a cell comprising contacting the cell with an effective amount of an anti-androgen drug. In some embodiments, the anti-androgen drug is an inhibitor of ABCB1 ATPase activity. In some cases, the anti-androgen drug comprises bicalutamide, enzalutamide, a derivative thereof, a salt thereof, or a combination thereof.

In a sixth aspect, the present invention provides a kit comprising a container containing a taxane and a container containing a drug that reduces efflux of the taxane from a cell. In some embodiments, the drug that reduces efflux is an inhibitor of ABCB1 efflux. In some embodiments, the drug that reduces efflux is an inhibitor of ABCB1 ATPase activity. In some embodiments, the drug that reduces efflux is an anti-androgen drug. In some cases, the anti-androgen drug comprises bicalutamide, enzalutamide, a derivative thereof, a salt thereof, or a combination thereof. In some cases, the anti-androgen drug is bicalutamide. In some cases, the anti-androgen drug is enzalutamide. In some cases, the taxane is selected from the group consisting of paclitaxel, docetaxel, cabazitaxel, taxol, hongdoushan A, hongdoushan B, hongdoushan C, baccatin I, baccatin II, a 10-deacetylbaccatin, and a combination thereof. In some cases, the taxane is docetaxel. In some cases, the container containing the taxane contains docetaxel and the container containing the drug that reduces efflux of the taxane from the cell contains bicalutamide, enzalutamide, a derivative thereof, a salt thereof, or a combination thereof. In some cases, the kit further comprises a label with instructions for administering the taxane and the drug that reduces efflux of the taxane from the cell.

### BRIEF DESCRIPTION OF THE DRAWINGS

FIGS. 1A-1D depict bicalutamide reversal of docetaxel resistance in TaxR cells. FIG. 1A: C4-2B and TaxR cells were seeded in 6-well plates at a density of 2×105 cells per well. TaxR cells were treated with 0.5 μM elacridar or 20 μM bicalutamide for 24 hrs. Cells were incubated with 1 μg/mL Rhodamine 123 for another 4 hrs. The cells were then washed 3 times with 1×PBS. Fluorescence was detected at an excitation wavelength of 480 nm and an emission wavelength of 534 nm. Top panel, fluorescent and phase contrast pictures are shown. Lower panel, rhodamine intake ratio. FIG. 1B: TaxR cells were plated in 12-well plates and treated with increasing concentrations of docetaxel in the presence or absence of 20 μM bicalutamide. Cell numbers were counted after 24 hrs of treatment. FIG. 1C: TaxR cells were treated with either 20 μmol/L bicalutamide or 10 nM docetaxel. After 24 hrs of treatment, cell number was counted (left panel). Whole-cell extracts were analyzed by western blot using specific antibodies as indicated (right panel). FIG. 1D: TaxR cells were treated with 10 nM docetaxel in the presence or absence of 20 μM bicalutamide. After 6 hrs of treatment, 1,000 cells were plated in 100 mm dishes in media containing complete FBS. The numbers of colonies were counted after 3 weeks and the results are presented as means±SD of 2 experiments performed in duplicate. **, P<0.01.

FIGS. 2A-2D depict enzalutamide reversal of docetaxel resistance in TaxR cells. FIG. 2A: TaxR cells were treated with either 20 μmol/L enzalutamide or with 10 nM docetaxel for 24 hrs. Cells were incubated with 1 μg/mL Rhodamine 123 for another 4 hrs. The cells were then washed three times with 1×PBS. Fluorescence was detected at an excitation wavelength of 480 nm and an emission wavelength of 534 nm. Fluorescent and phase contrast pictures are shown. Lower panel is the rhodamine intake ratio. FIG. 2B: TaxR cells were plated in 12-well plates and treated with increasing concentrations of docetaxel in the presence or absence of 20 μM enzalutamide. Cell numbers were counted after 24 hrs of treatment. FIG. 2C: TaxR cells were treated with either 20 μmol/L enzalutamide or with 10 nM docetaxel. After 24 hrs of treatment, the cell numbers were counted (left panel). Whole-cell extracts were analyzed by Western blot using specific antibodies as indicated (right panel). FIG. 2D: TaxR cells were treated with 10 nM docetaxel in the presence or absence of 20 μM enzalutamide. After 6 hrs of treatment, 1,000 cells were plated in 100 mm dishes in media containing complete FBS. The numbers of colonies were counted after 3 weeks. Results are presented as means±SD of 2 experiments performed in duplicate. **, P<0.01.

FIGS. 3A-3C depict the effects of bicalutamide and enzalutamide on ABCB1 ATPase activity. ABCB1 ATPase activity in response to 40 μM bicalutamide and 40 μM enzalutamide and 2.5 μM elacridar (FIG. 3A) or 50 μM docetaxel (FIG. 3B) is depicted. FIG. 3C: Effects of bicalutamide (10, 20 and 40 μM) and enzalutamide (10, 20 and 40 μM) on 50 μM docetaxel-induced ABCB1 ATPase activity are depicted. These values were normalized to the basal P-gp ATPase activity as described herein. **, P<0.01.

FIGS. 4A-4B illustrate that combination treatment with bicalutamide and docetaxel can overcome docetaxel resistance. Six to eight week-old SCID mice were inoculated s.c. with 4×106 C4-2B or TaxR cells on the flank. FIG. 4A: Mice carrying C4-2B tumors were divided into two groups to receive either vehicle or docetaxel (10 mg/kg body weight, i.p.) once a week. FIG. 4B: Mice carrying TaxR tumors were divided into four groups and treated with vehicle, docetaxel (10 mg/kg body weight, i.p., one day a week) or bicalutamide (25 mg/kg body weight, esophageal gavaging, 5 days a week) alone or a combination of docetaxel (10 mg/kg body weight, i.p., one day a week)+bicalutamide (25 mg/kg body weight, esophageal gavaging, 5 days a week). Tumor sizes were measured twice a week. *, P<0.05. **, P<0.01. DTX, docetaxel, Bic, bicalutamide.

FIGS. 5A-5E illustrate that bicalutamide can reverse docetaxel resistance in DU145-DTXR cells. FIG. 5A: DU145-DTXR cells were treated with 0.5 μM elacridar or 20 μM bicalutamide for 24 hrs. Cells were incubated with 1 μg/mL Rhodamine 123 for another 4 hrs. The cells were then washed 3 times with 1×PBS. Fluorescence was detected at an excitation wavelength of 480 nm and an emission wavelength of 534 nm. Top panel, fluorescent and phase contrast pictures are shown. Lower panel, rhodamine intake ratio. FIG. 5B: DU145 and DU145-DTXR cells were plated in 12-well plates. DU145 cells were treated with different concentrations of docetaxel as indicated. DU145-DTXR cells were treated with different concentrations of docetaxel as indicated in the presence or absence of 20 μM bicalutamide. Cell numbers were counted after 24 hrs of treatment. FIG. 5C: DU145-DTXR cells were plated in 12-well plates and treated with 10 nM docetaxel in the presence or absence of 20 μM bicalutamide. Cell numbers were counted after 24 hrs of treatment. FIG. 5D: Six to eight week-old SCID mice were inoculated s.c. with 4×10⁶ DU145-DTXR cells subcutaneously. Mice carrying tumors were divided into four groups and treated with vehicle, docetaxel (10 mg/kg body weight, i.p., one day a week) or bicalutamide (25 mg/kg body weight, esophageal gavaging, 5 days a week) alone or a combination of docetaxel (10 mg/kg body weight, i.p., one day a week)+bicalutamide (25 mg/kg body weight, esophageal gavaging, 5 days a week). Tumor sizes were measured twice a week. FIG. 5E: Ki67 was analyzed in tumor tissues by IHC staining and quantified as described herein. *, P<0.05. **, P<0.01.

FIGS. 6A-6C illustrate docetaxel resistance in DU145-DTXR cells. DU145 parental cells and DU145-DTXR cells were plated in 12-well plates at 1×10⁵ cells per well. Cells were treated with different concentrations of docetaxel for 48 hrs. FIG. 6A: Cell numbers were counted. FIG. 6B: Cell pictures were taken to show the cell survival inhibition. FIG. 6C: DU145 parental cells and DU145-DTXR cells were plated in 60 mm dishes at total RNA and protein lysates were collected after 24 hrs. ABCB1 is overexpressed in DU145-DTXR cells compared to DU145 parental cells in both mRNA and protein level (insertion panel).

FIGS. 7A-7C illustrate reversal of docetaxel resistance by knockdown of ABCB1 in DU145-DTXR cells. DU145-DTXR cells were plated in 12-well plate at 1×10⁵ cells per well. Cells were transfected with either vector control or specific shRNA against ABCB1. After 24 hrs of transfection, cells were treated with DMSO or 10 nM docetaxel for 48 hrs. FIG. 7A: Cell numbers were counted. FIG. 7B: Cell pictures were taken to show the cell survival inhibition. FIG. 7C: Total RNAs were collected from the cells after treatment, and were subjected to reverse transcription and realtime-PCR to verify ABCB1 knockdown by shRNA.

FIGS. 8A-8B illustrates reversal of docetaxel resistance in DU145-DTXR cells by contacting the cells with the ABCB1 activity inhibitor elacridar. DU145-DTXR cells were plated in 12-well plate at 1×10⁵ cells per well. Cells were treated with 10 nM docetaxel in the presence or absence of 0.5 μM elacridar for 48 hrs. FIG. 8A: Cell numbers were counted. FIG. 8B: Cell pictures were taken to show the cell survival inhibition.

FIGS. 9A-9B illustrate reversal of docetaxel resistance in DU145-DTXR cells by contacting the cells with anti-androgen drugs. DU145-DTXR cells were plated in 12-well plate at 1×10⁵ cells per well. FIG. 9A: Cells were treated with 10 nM docetaxel in the presence or absence of 20 μM bicalutamide (Bic) or enzalutamide (Enza) for 48 hrs. Cell numbers were counted (top panel: bicalutamide; bottom panel: enzalutamide). FIG. 9B: DU145 parental cells and DU145-DTX cells were plated in 12-well plate at 1×10⁵ cells per well. DU145 cells were treated with increasing concentrations of docetaxel. DU145-DTXR cells were treated with increasing concentrations of docetaxel in the presence or absence of 20 μM bicalutamide or enzalutamide for 48 hrs. Cell numbers were counted.

FIGS. 10A-10D illustrate that combination treatment with bicalutamide and docetaxel can overcome docetaxel resistance in C4-2B-docetaxel resistant cells (TaxR) in vivo. Six to eight week-old SCID mice were inoculated s.c. with 4×10⁶ C4-2B or TaxR cells on the flank. FIG. 10A: Mice carrying C4-2B tumors were divided into two groups to receive either vehicle or docetaxel (10 mg/kg body weight, i.p.) once a week. FIG. 10B: Mice carrying TaxR tumors were divided into four groups and treated with vehicle, docetaxel (10 mg/kg body weight, i.p., one day a week) or bicalutamide (25 mg/kg body weight, esophageal gavaging, 5 days a week) alone or a combination of docetaxel (10 mg/kg body weight, i.p., one day a week)+bicalutamide (25 mg/kg body weight, esophageal gavaging, 5 days a week). Tumor sizes were measured twice a week. ABCB1 expression was analyzed in tumor tissues by IHC staining (FIG. 10C) and western blotting (FIG. 10D). *, P<0.05. **, P<0.01. DTX, docetaxel, Bic, bicalutamide. Combi, docetaxel plus bicalutamide.

FIGS. 11A-11C illustrate that combination treatment with bicalutamide and docetaxel overcomes docetaxel resistance in DU145-DTXR cells in vivo. Six to eight week-old SCID mice were inoculated s.c. with 4×10⁶DU145-DTXR cells subcutaneously. FIG. 11A: Mice carrying tumors were divided into four groups and treated with vehicle, docetaxel (10 mg/kg body weight, i.p., one day a week) or bicalutamide (25 mg/kg body weight, esophageal gavaging, 5 days a week) alone or a combination of docetaxel (10 mg/kg body weight, i.p., one day a week)+bicalutamide (25 mg/kg body weight, esophageal gavaging, 5 days a week). Tumor sizes were measured twice a week. FIG. 11B: ABCB1 expression was analyzed in tumor tissues by western blot. FIG. 11C: Ki67 was analyzed in tumor tissues by IHC staining and quantified. *, P<0.05.

### DETAILED DESCRIPTION OF THE INVENTION

### I. Introduction

For patients with progressive castrate-resistant prostate cancer (CRPC) previously treated with docetaxel, both abiraterone acetate (Zytiga) and enzalutamide (Xtandi) confer a statistically significant improvement in overall survival. This led to the recent FDA approval of both agents. Abiraterone inhibits intra-tumoral androgen biosynthesis by blocking CYP17A1 (8). Enzalutamide impairs AR signaling by inhibiting AR nuclear translocation and DNA binding (9). These clinical results indicated that targeting AR signaling remains an important therapeutic strategy in docetaxel-resistant CRPC. Although both abiraterone and enzalutamide showed significant benefit to CRPC patients who failed docetaxel-based chemotherapy (both were also recently approved to treat CRPC patients prior to docetaxel treatment), these treatments are quite expensive. According to recent cost-effectiveness analysis, abiraterone treatment cost about $123.4K/quality-adjusted life year compared to placebo, and the cost of enzalutamide was $437.6K/quality-adjusted life year compared to abiraterone (10). The cost of these two drugs makes treating patients with metastatic castration-resistant prostate cancer an extremely expensive process, and more cost-effective therapeutic strategies are needed.

Numerous studies have uncovered potential mechanisms involved in the development of docetaxel resistance in prostate cancer. Accumulation of certain β-tubulin isotypes such as βIII-tubulin and βIV-tubulin in tumor cells has been shown to be associated with reduced response rate in patients receiving docetaxel-based chemotherapy (11, 12). β-tubulin mutations, such as T26A, A595G and F270I, have been demonstrated to impair tubulin polymerization in the presence of docetaxel in prostate cancer and breast cancer cell lines (13, 14). Alterations of cell survival factors and apoptosis regulators such as Bcl-2, clusterin, HSPs and IAPB are also observed in docetaxel-resistant prostate cancer cells (15-18). Aberrant activation of multiple cellular signaling pathways such as p53, NF-κB, PI3K-Akt and IL-6/STAT3 are associated with docetaxel resistance as well (13, 14, 19, 20). Additionally, recent studies also revealed that overexpressing Notch and Hedgehog signaling has high tumor-initiating capacity and the activation of GATA2-IGF axis confers taxane resistance in lethal prostate cancer (21, 22).

Our recent studies have also identified upregulation of ABCB1 as a common mechanism involved in acquired docetaxel resistance in CRPC (23). ABCB1 (P-glycoprotein, or MDR1) belongs to the ATP-binding cassette (ABC) transporters, a class of membrane transporters that use the energy produced during ATP hydrolysis to transport substrates, including taxanes such as docetaxel, across cell membranes and out of the cell. This diminishes the efficacy of the drug (24). Studies demonstrate that increased expression of ABCB1 confers resistance to chemotherapeutic agents (25-27). In addition, ABCB1 is overexpressed in many types of cancers including prostate, and ABCB1 expression is directly correlated with prostate tumor grade and stage (28).

As described herein, anti-androgens such as enzalutamide and bicalutamide can inhibit ABCB1 efflux activity and re-sensitize taxane-resistant prostate cancer cells to treatment with taxanes, such as docetaxel. For example, the previous-generation nonsteroidal anti-androgen, bicalutamide was able to overcome docetaxel resistance when combined with docetaxel in docetaxel-resistant prostate cancer cells both in vitro and in vivo. These results show that co-treatment with an anti-androgen such as bicalutamide, enzalutamide, a derivative thereof, a salt thereof, or a combination thereof, and a taxane, such as docetaxel, can be used as a combination therapy as an affordable regimen to treat docetaxel-resistant CRPC. These results are applicable to re-sensitize cancer cells other than prostate cancer cells that develop resistance to taxanes (e.g., docetaxel) by increasing the activity and/or expression of ABCB1.

### II. Definitions

As used herein, the following terms have the meanings ascribed to them unless specified otherwise.

It is noted here that as used in this specification and the appended claims, the singular forms “a,” “an,” and “the” include plural reference unless the context clearly dictates otherwise.

As used herein, the term “effective amount” includes a dosage sufficient to produce a desired result with respect to the indicated disorder, condition, or mental state. The desired result may comprise a subjective or objective improvement in the recipient of the dosage. For example, an effective amount of a taxane (e.g., docetaxel) or an anti-androgen drug (e.g., enzalutamide or bicalutamide) includes an amount sufficient to alleviate the signs, symptoms, or causes of cancer, e.g. taxane-resistant or docetaxel-resistant cancer. As another example, an effective amount of a taxane (e.g., docetaxel) or an anti-androgen drug (e.g., enzalutamide or bicalutamide) includes an amount sufficient to alleviate the signs, symptoms, or causes of prostate cancer, e.g. CRPC, taxane-resistant, or docetaxel-resistant prostate cancer.

Thus, an effective amount can be an amount that slows or reverses tumor growth, increases mean time of survival, inhibits tumor progression or metastasis, or re-sensitizes a cancer cell (e.g., prostate cancer cell) to a cancer drug (e.g., prostate cancer drug) to which it has become or is resistant (e.g., a taxane, such as docetaxel). Also, for example, an effective amount of a taxane, an anti-androgen drug, or a composition comprising a combination thereof includes an amount sufficient to cause a substantial improvement in a subject having cancer (e.g., prostate cancer) when administered to the subject. The effective mount can vary with the type and stage of the cancer being treated, the type and concentration of one or more compositions administered, and the amounts of other drugs that are also administered.

For example, the amount can vary with the type of prostate cancer being treated, the stage of advancement of the prostate cancer, the type and concentration of one or more compositions applied, and the amounts of other drugs that are also administered to the subject. An effective amount of anti-androgen drug (e.g., enzalutamide or bicalutamide) or an inhibitor of ABCB1 efflux activity (e.g., an inhibitor of ABCB1 ATPase activity) can include an amount that is effective in enhancing the anti-cancer (e.g., anti-prostate cancer) therapeutic activity of a taxane such as paclitaxel, docetaxel, cabazitaxel, taxol, hongdoushan A, hongdoushan B, hongdoushan C, baccatin I, baccatin II, or a 10-deacetylbaccatin.

As used herein, the term “treating” includes, but is not limited to, methods and manipulations to produce beneficial changes in a recipient's health status, e.g., a patient's cancer (e.g., prostate cancer) status. The changes can be either subjective or objective and can relate to features such as symptoms or signs of the cancer being treated. For example, if the patient notes decreased pain, then successful treatment of pain has occurred. For example, if a decrease in the amount of swelling has occurred, then a beneficial treatment of inflammation has occurred. Similarly, if the clinician notes objective changes, such as reducing the number of cancer cells, the growth of the cancer cells, the size of cancer tumors, or the resistance of the cancer cells to another cancer drug (e.g., a taxane such as docetaxel), then treatment of cancer has also been beneficial. Preventing the deterioration of a recipient's status is also included by the term. Treating, as used herein, also includes administering a taxane (e.g., docetaxel), an inhibitor of taxane efflux (for example, an inhibitor of ABCB1 efflux or ATPase activity of an ABC transporter such as ABCB1, such as an anti-androgen drug (e.g., enzalutamide or bicalutamide)), or a combination thereof to a patient having cancer (e.g., prostate cancer).

As used herein, the term “administering” includes activities associated with providing a patient an amount (e.g., an effective amount) of a compound or composition described herein, e.g., a taxane (e.g., docetaxel), an inhibitor of taxane efflux (e.g., enzalutamide or bicalutamide), or a combination thereof. Administering includes providing unit dosages of compositions set forth herein to a patient in need thereof. Administering includes providing effect amounts of compounds or compositions described herein for specified period of time, e.g., for about 1, 2, 3, 4, 5, 6, 7, 8, 9, 10, 12, 13, 14, 15, 16, 17, 18, 19, 20, 21, 22, 23, 24, 25, 26, 27, 28, 29, 30, or 31 days, or in a specified sequence, e.g., administration of a taxane (e.g., docetaxel) followed by the administration of a composition that inhibits taxane efflux (e.g., an anti-androgen such as enzalutamide, bicalutamide, or a combination thereof), or vice versa.

As used herein, the term “co-administering” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. The two or more structurally different compounds can be comprised of a taxane (e.g., docetaxel) and an inhibitor of taxane efflux (e.g., an ABCB1 efflux inhibitor such as one or both of the anti-androgens enzalutamide or bicalutamide). In some instances, the co-administered compounds are administered by the same route. In other instances, the co-administered compounds are administered via different routes. For example, one compound can be administered orally, and the other compound can be administered, e.g., sequentially or simultaneously, via intravenous or intraperitoneal injection. The simultaneously or sequentially administered compounds or compositions can be administered such that at least one taxane and at least one ABCB1 efflux inhibitor (e.g., enzalutamide or bicalutamide) are simultaneously present in a subject or in a cell at an effective concentration.

As used herein, the term “cancer” refers to conditions including solid cancers, lymphomas, and leukemias. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma, head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia, and multiple myeloma. The cancer can be a drug-resistant cancer, such as a taxane-resistant cancer. The cancer can be a docetaxel-resistant cancer. The cancer can be a taxane re-sensitized cancer, such as a cancer that is re-sensitized to taxane (e.g., docetaxel) by contact with an ABC transport inhibitor (e.g., an ABCB1 inhibitor such as enzalutamide or bicalutamide).

As used herein, the terms “prostate cancer” or “prostate cancer cell” refer to a cancer cell or cells that reside in prostate tissue. The prostate cancer can be benign, malignant, or metastatic. The prostate cancer can be androgen-insensitive, hormone-resistant, or castrate-resistant. The prostate cancer can be “advanced stage prostate cancer” or “advanced prostate cancer.” Advanced stage prostate cancer includes a class of prostate cancers that has progressed beyond early stages of the disease. Typically, advanced stage prostate cancers are associated with a poor prognosis. Types of advanced stage prostate cancers include, but are not limited to, metastatic prostate cancer, drug-resistant prostate cancer such as anti-androgen-resistant prostate cancer (e.g., enzalutamide-resistant prostate cancer, taxane-resistant prostate cancer, docetaxel-resistant prostate cancer, abiraterone-resistant prostate cancer, bicalutamide-resistant prostate cancer, and the like), hormone refractory prostate cancer, castrate-resistant prostate cancer, metastatic castrate-resistant prostate cancer, docetaxel-resistant prostate cancer, and combinations thereof. In some instances, the advanced stage prostate cancers do not generally respond, or are resistant, to treatment with one or more of the following conventional prostate cancer therapies: enzalutamide, abiraterone, bicalutamide, and a taxane such as docetaxel. In some instances, the advanced stage prostate cancers do not generally respond, or are resistant, to treatment with a taxane such as docetaxel. In some instances, the advanced stage prostate cancers do not generally respond, or are resistant, to treatment with docetaxel. Compounds, compositions, and methods of the present invention are provided for treating prostate cancer, such as advanced stage prostate cancer, including any one or more (e.g., two, three, four, five, six, seven, eight, nine, ten, or more) of the types of advanced stage prostate cancers disclosed herein.

As used herein, the phrase “ameliorating the symptoms of prostate cancer” includes alleviating or improving the symptoms or condition of a patient having cancer (e.g., prostate cancer). Ameliorating the symptoms includes reducing the pain or discomfort associated with cancer. Ameliorating the symptoms also includes reducing the markers of cancer, e.g., reducing the number of cancer cells or reducing the size of cancer tumors.

As used herein, the phrase “enhancing the therapeutic effects” includes any of a number of subjective or objective factors indicating a beneficial response or improvement of the condition being treated as discussed herein. For example, enhancing the therapeutic effects of a taxane (e.g., docetaxel) includes re-sensitizing taxane (e.g., docetaxel)-resistant cancer (e.g., prostate cancer) to taxane (e.g., docetaxel) therapy. Also, for example, enhancing the therapeutic effects of a taxane (e.g., docetaxel) includes altering taxane (e.g., docetaxel)-resistant cancer cells (e.g., prostate cancer cells) so that the cells are not resistant to the taxane (e.g., docetaxel). Also, for example, enhancing the therapeutic effects of a taxane (e.g., docetaxel) includes additively or synergistically improving or increasing the activity of the taxane (e.g., docetaxel). In some embodiments, the enhancement includes, or includes at least, a one-fold, two-fold, three-fold, five-fold, ten-fold, twenty-fold, fifty-fold, hundred-fold, or thousand-fold increase in the therapeutic activity of the taxane (e.g., docetaxel) used to treat cancer (e.g., prostate cancer). In some embodiments, the enhancement includes, or includes at least, a 10%, 20%, 30%, 40%, 50%, 60%, 75%, 80%, 90%, or 100% increase in the therapeutic activity (e.g., efficacy) of the taxane (e.g., docetaxel) used to treat cancer (e.g., prostate cancer).

As used herein, the terms “reversing cancer cell resistance,” “reducing cancer cell resistance,” or “re-sensitizing cancer cell resistance” to a taxane includes altering or modifying a cancer cell that is resistant to a taxane (e.g., docetaxel) therapy so that the cell is no longer resistant to taxane (e.g., docetaxel) therapy, or is less resistant to the taxane therapy. As such, as used herein, the phrase “reversing prostate cancer cell resistance” to a taxane includes altering or modifying a prostate cancer cell that is resistant to a taxane (e.g., docetaxel) therapy so that the cell is no longer resistant to taxane (e.g., docetaxel) therapy, or is less resistant to the taxane therapy.

As used herein, the phrase “anti-androgen drug” includes anti-androgen compounds that alter the androgen pathway by blocking the androgen receptors, competing for binding sites on the cell's surface, or affecting or mediating androgen production. Anti-androgens are useful for treating several diseases including, but not limited to, cancer (e.g., prostate cancer). Anti-androgens include, but are not limited to, enzalutamide, bicalutamide, arbiraterone, nilutamide, flutamide, apalutamide, cyproterone acetate, megestrol acetate, chlormadinone acetate, spironolactone, canrenone, drospirenone, topilutamide, cimetidine, finasteride, dutasteride, alfatradiol, derivatives thereof, salts thereof, and combinations thereof. In some cases, the anti-androgens are non-steroidal anti-androgens that bind and antagonize androgen receptor signaling. In some cases, the anti-androgens are enzalutamide, bicalutamide, nilutamide, flutamide, apalutamide, cyproterone acetate, megestrol acetate, chlormadinone acetate, spironolactone, canrenone, drospirenone, topilutamide, cimetidine, a derivative thereof, a salt thereof, or a combination thereof. In some cases, the anti-androgen drugs are enzalutamide, bicalutamide, a derivative of enzalutamide or bicalutamide, or a combination thereof. Derivatives of, e.g., enzalutamide, bicalutamide, nilutamide, flutamide, apalutamide, cyproterone acetate, megestrol acetate, chlormadinone acetate, spironolactone, canrenone, drospirenone, topilutamide, or cimetidine, that reduce ABCB1-mediated efflux of taxanes in a cell can be identified by contacting the cell with a candidate anti-androgen derivative of enzalutamide or bicalutamide and measuring ABCB1 activity using an ABCB1 activity assay such as the Pgp-Glo™ Assay System.

As used herein, the term “taxane” refers to diterpene compounds that contain a taxadiene core. Exemplary taxanes include, but are not limited to, paclitaxel, docetaxel, cabazitaxel, taxol, hongdoushan A, hongdoushan B, hongdoushan C, baccatin I, baccatin II, and the 10-deacetylbaccatins (e.g., 7-Epi-10-Deacetylbaccatin III, 10-Deacetylbaccatin V, 10-Deacetylbaccatin III, 10-Deacetylbaccatin VI, 13-Epi-10-Deacetylbaccatin III, 13-[3-(2-Naphthyl)prop-2-enoyl]-2-debenzoyl-2-(4-methoxybenzoxyl)-10-deacetylbaccatin III, and 10-N,N-Dimethylglycyl-13-[3-(2-Naphthyl)prop-2-enoyl]-10-deacetylbaccatin III), and combinations thereof.

The term “individual,” “subject,” or “patient” typically includes humans, but also includes other animals such as, e.g., other primates, rodents, canines, felines, equines, ovines, porcines, and the like.

“Pharmaceutically acceptable” or “therapeutically acceptable” includes a substance which does not interfere with the effectiveness or the biological activity of the active ingredients and which is not toxic to the hosts in the amounts used, and which hosts may be either humans or animals to which it is to be administered.

### III. Compositions

Certain cancers can be treated (e.g., contacted with a substance to inhibit growth, proliferation, viability, migration, or metastasis) with compounds or compositions that disrupt androgen receptor signaling. Such compounds that disrupt androgen receptor signaling include anti-androgen drugs. Anti-androgen drugs include steroidal anti-androgen drugs and non-steroidal anti-androgen drugs. Non-steroidal anti-androgen drugs include bicalutamide, enzalutamide, or derivatives thereof. The anti-androgen drugs can be selective androgen receptor inhibitors, such as selective androgen receptor modulators (SARM) that act as partial or complete antagonists of androgen receptor signaling.

In some cases, the anti-androgen drugs are inhibitors of an efflux mechanism in a cell. For example, the anti-androgen drug can inhibit a plasma membrane bound pump that actively transports small organic molecules out of the cell. In some cases, the anti-androgen drug is an inhibitor of an ATP-dependent efflux pump. In some cases, the anti-androgen drug inhibits the ATP-dependent efflux pump by inhibiting the ATPase activity of the efflux pump. In some cases, the anti-androgen drug is an inhibitor of the ATP-dependent efflux pump ABCB1. In some cases, the anti-androgen drug is an inhibitor of the ATPase activity of ABCB1. Inhibition of ABCB1 efflux activity and/or ATPase activity can increase the effectiveness of one or more drugs in a cell that would otherwise actively transport the one or more drugs out of the cell. In some cases, the anti-androgen drugs (e.g., enzalutamide, bicalutamide, or a combination thereof) can inhibit ATP-dependent efflux (e.g., by inhibiting ABCB1 activity) in an androgen receptor independent manner. Thus, the anti-androgen drugs can inhibit efflux in an AR-positive or AR-negative cell (e.g., an AR-positive or AR-negative cancer cell such as an AR-positive prostate cancer cell or AR-negative prostate cancer cell).

In some cases, upregulation, dysregulation, or changes in substrate specificity, activity, or enzyme kinetics (e.g., through mutations in the primary sequence or a regulatory region of a protein involved in the efflux mechanism of a cell) can lead to resistance of a cell to one or more drugs. For example, in some cases, an increase in ABCB1 activity, amount, or expression can lead to an increase in resistance of a cancer cell (e.g., prostate cancer cell) to a taxane (e.g., docetaxel). In such cases, the resistance can be overcome, reduced, or reversed, by inhibiting the efflux mechanism, such as with an anti-androgen drug.

In some cases, a cancer (e.g., prostate cancer) can be treated with a taxane. Taxanes are a class of terpene-based microtubule disruptors derived from or structurally related to taxol. Taxanes include but are not limited to paclitaxel, docetaxel, cabazitaxel, taxol, hongdoushan A, hongdoushan B, hongdoushan C, baccatin I, or baccatin II. Taxanes can further include, but are not limited to 10-deacetylbaccatins, or derivatives thereof, such as 10-Deacetylbaccatin III, 10-Deacetylbaccatin V, 10-Deacetylbaccatin VI, 13-Epi-10-Deacetylbaccatin III, 13-[3-(2-Naphthyl)prop-2-enoyl]-2-debenzoyl-2-(4-methoxybenzoxyl)-10-deacetylbaccatin III, or 10-N,N-Dimethylglycyl-13-[3-(2-Naphthyl)prop-2-enoyl]-10-deacetylbaccatin III. In some cases, a cancer cell that has developed resistant to a taxane (e.g., docetaxel) can be treated with an ABCB1 inhibitor such as an anti-androgen drug (e.g., bicalutamide, enzalutamide) to reduce, reverse, or overcome the taxane resistance.

In any of the compositions described herein, the composition may further include a pharmaceutically acceptable excipient or diluent.

### IV. Pharmaceutical Formulations

The pharmaceutical formulations of the present invention encompass formulations made by admixing a taxane such as docetaxel, and a pharmaceutically acceptable carrier and/or excipient or diluent. Such formulations are suitable for pharmaceutical use in an animal or human.

The pharmaceutical compositions of the present invention also encompass compositions made by admixing an inhibitor of taxane efflux from a cell, such as an inhibitor of ABCB1-mediated efflux of taxane from the cell, and a pharmaceutically acceptable carrier and/or excipient or diluent. The inhibitor of taxane efflux can be an inhibitor of ABCB1 ATPase activity. In some cases, the inhibitor of taxane efflux is an inhibitor of docetaxel efflux. In some cases, the inhibitor of taxane efflux is an anti-androgen drug. In some cases, the anti-androgen drug contains enzalutamide, bicalutamide, or a combination thereof. Such formulations are suitable for pharmaceutical use in an animal or human.

The pharmaceutical compositions of the present invention also encompass compositions made by admixing an inhibitor of taxane efflux from a cell, a taxane, and a pharmaceutically acceptable carrier and/or excipient or diluent. For example, the pharmaceutical composition can be made by admixing an anti-androgen drug such as enzalutamide, bicalutamide, or a combination thereof, a taxane such as docetaxel, and a pharmaceutically acceptable carrier and/or excipient or diluent. Such formulations are suitable for pharmaceutical use in an animal or human. The pharmaceutical formulations of the present invention can include drug, e.g., enzalutamide, bicalutamide, and/or docetaxel, or any pharmaceutically acceptable salts thereof, as an active ingredient and a pharmaceutically acceptable carrier and/or excipient or diluent. A pharmaceutical composition may optionally contain other therapeutic ingredients.

The pharmaceutical formulations of the present invention may be prepared by any of the methods well-known in the art of pharmacy. Pharmaceutically acceptable carriers suitable for use with the present invention include any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent.

The compounds of the present invention can be combined as the active ingredient in intimate admixture with a suitable pharmaceutical carrier and/or excipient according to conventional pharmaceutical compounding techniques. Any carrier and/or excipient suitable for the form of preparation desired for administration is contemplated for use with the compounds disclosed herein. The formulations include formulations suitable for topical, parenteral, pulmonary, nasal, rectal, or oral administration. Other preferred formulations include formulations suitable for systemic (enteral or parenteral) administration. Systemic administration includes oral, rectal, sublingual, or sublabial administration. In some embodiments, the compositions may be administered via a syringe or intravenously. The most suitable route of administration in any given case will depend in part on the nature and severity of the cancer (e.g., prostate cancer) condition and also optionally the stage of the cancer.

Formulations for pulmonary administration include, but are not limited to, dry powder formulations containing a powder form of one or more compounds described herein (e.g., a powder form of a taxane and an inhibitor of taxane efflux), or a salt thereof, and the powder of a suitable carrier and/or lubricant. The formulations for pulmonary administration can be inhaled from any suitable dry powder inhaler device known to a person skilled in the art. Formulations for pulmonary administration include, but are not limited to, liquid formulations one or more compounds described herein (e.g., a taxane and an inhibitor of taxane efflux), or a salt thereof, and a suitable liquid carrier and/or lubricant. The liquid formulations for pulmonary administration can be inhaled from any suitable aerosol or nebulizer inhaler device known to a person skilled in the art.

Formulations for systemic administration include, but are not limited to, dry powder formulations containing a powder form of one or more compounds described herein (e.g., a powder form of a taxane and an inhibitor of taxane efflux), or a salt thereof, and the powder of a suitable carrier and/or lubricant. The powder formulation can be diluted into a suitable diluent to form a syrup, solution, or suspension. The oral formulation for systemic administration can be formed into, and/or delivered in the form of, a tablet, capsule, pill, syrup, solution, or suspension.

In some embodiments, the present invention provides formulations further including a pharmaceutical surfactant. In other embodiments, the present invention provides formulations further including a cryoprotectant. In some embodiments, the cryoprotectant is selected from the group consisting of glucose, sucrose, trehalose, lactose, sodium glutamate, PVP, HPβCD, CD, glycerol, maltose, mannitol, and saccharose.

Pharmaceutical formulations or medicaments for use in the present invention can be formulated by standard techniques using one or more physiologically acceptable carriers or excipients. Suitable pharmaceutical carriers are described herein and in Remington: The Science and Practice of Pharmacy, 21st Ed., University of the Sciences in Philadelphia, Lippencott Williams & Wilkins (2005).

Controlled release parenteral formulations of one or more compounds or compositions of the present invention can be made as implants, oily injections, or as particulate systems. For a broad overview of delivery systems see, Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995) incorporated herein by reference. Particulate systems include microspheres, microparticles, microcapsules, nanocapsules, nanospheres, and nanoparticles.

Polymers can be used in a pharmaceutical formulation for ion-controlled release of one or more compounds or compositions of the present invention. Various degradable and nondegradable polymeric matrices for use in controlled drug delivery are known in the art (Langer R., Accounts Chem. Res., 26:537-542 (1993)). For example, the block copolymer, polaxamer 407 exists as a viscous yet mobile liquid at low temperatures but forms a semisolid gel at body temperature. It has shown to be an effective vehicle for formulation and sustained delivery of recombinant interleukin 2 and urease (Johnston et al., Pharm. Res., 9:425-434 (1992); and Pec et al., J. Parent. Sci. Tech., 44(2):58 65 (1990)). Alternatively, hydroxyapatite has been used as a microcarrier for controlled release of proteins (Ijntema et al., Int. J. Pharm., 112:215-224 (1994)). In yet another aspect, liposomes are used for controlled release as well as drug targeting of the lipid-capsulated drug (Betageri et al., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

### V. Methods of Treating Cancer

In some embodiments, the present invention provides a method of treating cancer in a patient (e.g., a prostate cancer, such as a taxane-resistant prostate cancer), wherein the method comprises administering to the patient an effective amount of a taxane and a drug that reduces efflux of the taxane from the cell (e.g., an inhibitor of taxane efflux). The taxane can be any one of paclitaxel, docetaxel, cabazitaxel, taxol, hongdoushan A, hongdoushan B, hongdoushan C, baccatin I, baccatin II, a 10-deacetylbaccatin, or a derivative or combination thereof. In some cases, the taxane is docetaxel. In some cases, the effective amount of the taxane administered simultaneously or sequentially with the drug that reduces efflux of taxane is less than the amount of taxane administered to treat the cancer in the absence of the drug that reduces efflux of taxane. The reduction in the effective amount of the taxane can be (at least) about a 1%, 5%, 10%, 15%, 20%, 25%, 30%, 35%, 40%, 45%, 50%, 55%, 60%, 65%, 70%, 75%, 80%, 85%, 90%, 95%, or greater reduction in the effective amount of the taxane. In some cases, the taxane is not effective in the absence of the drug that reduces efflux of taxane at doses or dose ranges that are practical to administer to a subject.

In some cases, the method comprises administering a taxane and a drug that inhibits ABCB1 efflux activity (e.g., by inhibiting ABCB1 ATPase activity). In some cases, the drug that reduces taxane efflux is an anti-androgen drug. In some cases, the anti-androgen drug is selected from the group consisting of a drug that contains enzalutamide, a drug that contains bicalutamide, and a drug that contains a combination of enzalutamide and bicalutamide. In some cases, the anti-androgen drug (e.g., enzalutamide and/or bicalutamide) is an inhibitor of ABCB1 efflux activity or ABCB1 ATPase activity. As such, the method can include administering docetaxel and enzalutamide or bicalutamide. Additionally, or alternatively, the method can include administering docetaxel and enzalutamide. As yet another alternative, the method can include administering docetaxel and bicalutamide. As yet another alternative, the method can include administering docetaxel and a combination of enzalutamide and bicalutamide.

In some of these embodiments, the cancer is a prostate cancer, such as advanced stage prostate cancer. The advanced stage prostate cancer can be any one or more of the types of advanced staged prostate cancers disclosed herein. In some of these embodiments, the prostate cancer is drug resistant. In some of these embodiments, the prostate cancer is resistant to a taxane (e.g., docetaxel). In some of these embodiments, the prostate cancer is metastatic. In some of these embodiments, the prostate cancer is metastatic and drug-resistant (e.g., taxane resistant). In some of these embodiments, the prostate cancer is castrate-resistant. In some of these embodiments, the prostate cancer is metastatic and castrate-resistant. In some of these embodiments, the prostate cancer is docetaxel resistant. In some of these embodiments, the prostate cancer is selected from the group consisting of metastatic prostate cancer, castrate-resistant prostate cancer, castration recurrent prostate cancer, androgen insensitive prostate cancer, hormone-resistant prostate cancer, and metastatic castrate-resistant prostate cancer.

In some embodiments, the treating comprises inhibiting cancer cell growth; inhibiting cancer cell migration; inhibiting cancer cell invasion; ameliorating the symptoms of cancer; reducing the size of a cancer tumor; reducing the number of cancer tumors; reducing the number of cancer cells; inducing cancer cell necrosis, pyroptosis, oncosis, apoptosis, autophagy, or other cell death; or enhancing the therapeutic effects of a taxane, such as docetaxel. In some embodiments, the treating comprises inhibiting prostate cancer cell growth; inhibiting prostate cancer cell migration; inhibiting prostate cancer cell invasion; ameliorating the symptoms of prostate cancer; reducing the size of a prostate cancer tumor; reducing the number of prostate cancer tumors; reducing the number of prostate cancer cells; inducing prostate cancer cell necrosis, pyroptosis, oncosis, apoptosis, autophagy, or other cell death; or enhancing the therapeutic effects of a taxane, such as docetaxel.

In some methods of treating cancer (e.g., prostate cancer), described herein, the treating comprises inhibiting cancer (e.g., prostate cancer) cell growth. In some methods of treating cancer (e.g., prostate cancer), described herein, the treating comprises inhibiting cancer (e.g., prostate cancer) cell migration. In some methods of treating cancer (e.g., prostate cancer), described herein, the treating comprises inhibiting cancer (e.g., prostate cancer) cell invasion. In some methods of treating cancer (e.g., prostate cancer), described herein, the treating comprises ameliorating the symptoms of cancer (e.g., prostate cancer). In some methods of treating cancer (e.g., prostate cancer), described herein, the treating comprises reducing the size of a cancer (e.g., prostate cancer) tumor. In some methods of treating cancer (e.g., prostate cancer), described herein, the treating comprises reducing the number of cancer (e.g., prostate cancer) tumors. In some methods of treating cancer (e.g., prostate cancer), described herein, the treating comprises reducing the number of cancer (e.g., prostate cancer) cells. In some methods of treating cancer (e.g., prostate cancer), described herein, the treating comprises inducing cancer (e.g., prostate cancer) cell necrosis, pyroptosis, oncosis, apoptosis, autophagy, or other cell death.

In particular methods of treating cancer (e.g., prostate cancer), described herein, the treating comprises enhancing the therapeutic effects of a taxane such as docetaxel. The enhancement can be synergistic or additive. In some cases, the enhancing the therapeutic effects of a taxane include reducing or reversing resistance of a cancer (e.g., prostate cancer) to the taxane, or re-sensitizing the cancer (e.g., prostate cancer) to taxane. In some case, the reducing, reversing, or re-sensitizing includes reducing efflux of the taxane (e.g., docetaxel) from the cancer (e.g., prostate cancer) cells. In some cases, the efflux of the taxane from the cancer (e.g., prostate cancer) cells is performed by contacting the cells with an inhibitor of taxane efflux (e.g., an ABCB1 efflux inhibitor, an ABCB1 ATPase inhibitor, an anti-androgen drug such as enzalutamide, bicalutamide, or a combination thereof).

### VI. Administration

In some embodiments of the present invention, a taxane, such as docetaxel, is administered to a patient having cancer (e.g., prostate cancer).

In some embodiments of the present invention, an anti-androgen drug, such as enzalutamide, bicalutamide, or a combination thereof, is administered in combination with the taxane (e.g., docetaxel).

In some embodiments of the present invention, an inhibitor of ABCB1 efflux activity and/or ATPase activity, is administered in combination with the taxane (e.g., docetaxel).

In certain methods of treating cancer (e.g., prostate cancer), set forth herein, the methods comprise first administering a taxane, such as docetaxel, to a patient having cancer (e.g., prostate cancer), and then administering an inhibitor of taxane efflux to the patient. In certain methods of treating cancer (e.g., prostate cancer), set forth herein, the methods comprise first administering an inhibitor of taxane efflux to a patient having cancer (e.g., prostate cancer), and then administering a taxane, such as docetaxel, to the patient.

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E. W. Martin. Compounds and agents of the present invention and their physiologically acceptable salts and solvates can be formulated for administration by any suitable route, including via inhalation, topically, nasally, orally, intravenously, parenterally, or rectally.

a. Routes of Administration

Typical formulations for topical administration include creams, ointments, sprays, lotions, and patches. The pharmaceutical composition can, however, be formulated for any type of administration, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral or rectal administration is also contemplated.

Suitable formulations for transdermal application include an effective amount of one or more compositions or compounds described herein, optionally with a carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

For oral administration, a pharmaceutical formulation or a medicament can take the form of, for example, a tablet or a capsule prepared by conventional means with a pharmaceutically acceptable excipient. The present invention provides tablets and gelatin capsules comprising a taxane (e.g., docetaxel), and/or an inhibitor of taxane efflux, such as an inhibitor of ABCB1 efflux and/or ATPase activity (e.g., an anti-androgen drug such as bicalutamide or enzalutamide), alone or in combination with other compounds, or a dried solid powder of these drugs, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, talcum, stearic acid, magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound(s).

The compositions and formulations set forth herein can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient(s) can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient(s).

For administration by inhalation, the compositions of the present invention may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compositions set forth herein can also be formulated in rectal compositions, for example, suppositories or retention enemas, for example, containing conventional suppository bases, for example, cocoa butter or other glycerides.

Furthermore, the active ingredient(s) can be formulated as a depot preparation. Such long-acting formulations can be administered by implantation (for example, subcutaneously or intramuscularly) or by intramuscular injection. Thus, for example, one or more of the compounds described herein can be formulated with suitable polymeric or hydrophobic materials (for example as an emulsion in an acceptable oil) or ion exchange resins, or as sparingly soluble derivatives, for example, as a sparingly soluble salt.

In particular embodiments, a pharmaceutical composition or medicament of the present invention can comprise (i) an effective amount of a taxane (e.g., docetaxel), and/or (ii) an effective amount of an inhibitor of taxane efflux, such as an inhibitor of ABCB1 efflux and/or ATPase activity (e.g., an anti-androgen drug such as bicalutamide or enzalutamide), alone or in combination with other compounds. The therapeutic agent(s) may be used individually, sequentially, or in combination with one or more other such therapeutic agents (e.g., a first therapeutic agent, a second therapeutic agent, a compound of the present invention, etc.). Administration may be by the same or different route of administration or together in the same pharmaceutical formulation.

b. Dosage

Pharmaceutical compositions or medicaments can be administered to a subject at a therapeutically effective dose to prevent, treat, re-sensitize, or control cancer (e.g., prostate cancer) as described herein. The pharmaceutical composition or medicament is administered to a subject in an amount sufficient to elicit an effective therapeutic response in the subject.

The dosage of active agents administered is dependent on the subject's body weight, age, individual condition, surface area or volume of the area to be treated and on the form of administration. The size of the dose also will be determined by the existence, nature, and extent of any adverse effects that accompany the administration of a particular formulation in a particular subject. A unit dosage for oral administration to a mammal of about 50 to about 70 kg may contain between about 5 and about 500 mg, about 25-200 mg, about 100 and about 1000 mg, about 200 and about 2000 mg, about 500 and about 5000 mg, or between about 1000 and about 2000 mg of the active ingredient. A unit dosage for oral administration to a mammal of about 50 to about 70 kg may contain about 10 mg, 20 mg, 25 mg, 50 mg, 75 mg, 100 mg, 200 mg, 300 mg, 400 mg, 500 mg, 600 mg, 700 mg, 800 mg, 900 mg, 1,000 mg, 1,250 mg, 1,500 mg, 2,000 mg, 2,500 mg, 3,000 mg, or more of the active ingredient. Typically, a dosage of the active compound(s) of the present invention is a dosage that is sufficient to achieve the desired effect. Optimal dosing schedules can be calculated from measurements of active agent accumulation in the body of a subject. In general, dosage may be given once or more of daily, weekly, or monthly. Persons of ordinary skill in the art can easily determine optimum dosages, dosing methodologies and repetition rates.

Optimum dosages, toxicity, and therapeutic efficacy of the compositions of the present invention may vary depending on the relative potency of the administered composition and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

Optimal dosing schedules can be calculated from measurements of active ingredient accumulation in the body of a subject. In general, dosage is from about 1 ng to about 1,000 mg per kg of body weight and may be given once or more daily, weekly, monthly, or yearly. Persons of ordinary skill in the art can easily determine optimum dosages, dosing methodologies and repetition rates. One of skill in the art will be able to determine optimal dosing for administration of taxanes, such as docetaxel, or taxane efflux inhibitors, such as inhibitors of ABCB1 efflux and/or ATPase activity (e.g., anti-androgen drugs, such as enzalutamide or bicalutamide), to a human being following established protocols known in the art and the disclosure herein.

The data obtained from, for example, animal studies (e.g. rodents and monkeys) can be used to formulate a dosage range for use in humans. The dosage of compounds of the present invention lies preferably within a range of circulating concentrations that include the ED₅₀ with little or no toxicity. The dosage can vary within this range depending upon the dosage form employed and the route of administration. For any composition for use in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a chimeric protein, preferably a composition is from about 1 ng/kg to about 100 mg/kg for a typical subject. In some embodiments, a single oral dose of 5 mg/kg niclosamide in rats can generate a maximal plasma concentration of 1.08 μmol/mL

A typical composition of the present invention for oral or intravenous administration can be about 0.1 to about 10 mg of active ingredient per patient per day; about 1 to about 100 mg per patient per day; about 25 to about 200 mg per patient per day; about 50 to about 500 mg per patient per day; about 100 to about 1000 mg per patient per day; or about 1000 to about 2000 mg per patient per day. Exemplary dosages include, but are not limited to, about 10 mg, 20 mg, 25 mg, 50 mg, 75 mg, 100 mg, 200 mg, 300 mg, 400 mg, 500 mg, 600 mg, 700 mg, 800 mg, 900 mg, 1,000 mg, 1,250 mg, 1,500 mg, 2,000 mg, 2,500 mg, 3,000 mg, or more of the active ingredient per patient per day. Methods for preparing administrable compositions will be known or apparent to those skilled in the art and are described in more detail in such publications as Remington: The Science and Practice of Pharmacy, 21st Ed., University of the Sciences in Philadelphia, Lippencott Williams & Wilkins (2005).

Exemplary doses of the compositions described herein include milligram or microgram amounts of the composition per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a composition depend upon the potency of the composition with respect to the desired effect to be achieved. When one or more of these compositions is to be administered to a mammal, a physician, veterinarian, or researcher may, for example, prescribe a relatively low dose at first, subsequently increasing the dose until an appropriate response is obtained. In addition, it is understood that the specific dose level for any particular mammal subject will depend upon a variety of factors including the activity of the specific composition employed, the age, body weight, general health, gender, and diet of the subject, the time of administration, the route of administration, the rate of excretion, any drug combination, and the degree of expression or activity to be modulated.

In some embodiments, a pharmaceutical composition or medicament of the present invention is administered, e.g., in a daily dose in the range from about 1 mg of compound per kg of subject weight (1 mg/kg) to about 1 g/kg. In another embodiment, the dose is a dose in the range of about 5 mg/kg to about 500 mg/kg. In yet another embodiment, the dose is about 10 mg/kg to about 250 mg/kg. In another embodiment, the dose is about 25 mg/kg to about 150 mg/kg. A preferred dose is about 2, 3, 4, 5, 6, 7, 8, 9, 10, 12, 15, 18, 20, 25, 30, 40, or 50 mg/kg. The daily dose can be administered once per day or divided into subdoses and administered in multiple doses, e.g., twice, three times, or four times per day. However, as will be appreciated by a skilled artisan, compositions described herein may be administered in different amounts and at different times. The skilled artisan will also appreciate that certain factors may influence the dosage and timing required to effectively treat a subject, including but not limited to the severity of the disease or malignant condition, previous treatments, the general health and/or age of the subject, and other diseases present. Moreover, treatment of a subject with a therapeutically effective amount of a composition can include a single treatment or, preferably, can include a series of treatments.

To achieve the desired therapeutic effect, compounds or agents described herein may be administered for multiple days at the therapeutically effective daily dose. Thus, therapeutically effective administration of compounds to treat cancer (e.g., prostate cancer) in a subject may require periodic (e.g., daily) administration that continues for a period ranging from three days to two weeks or longer. Compositions set forth herein may be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every other day, every third day, or, if higher dose ranges are employed and tolerated by the subject, once a week, once every two weeks, once every three weeks, once every four weeks, or even less frequently.

In some embodiments, the taxane docetaxel is orally administered. In some embodiments, the docetaxel is orally administered to a subject (e.g., an adult human) at a daily dose of approximately 1, 2, 3, 4, 5, 6, 7, 8, 9, 10, 11, 12, 15, 20, 25, 30, 40, 50, 75, 100; 200; 300; 400; 500; 600; 700; 800; 900; 1,000; 1,250; 1,500; 1,750; 2,000; 2,500; 3,000; 3,500; 4,000; 4,500; 5,000; or more mg of docetaxel per day. In some embodiments, the docetaxel is orally administered to a subject (e.g., an adult human) at a daily dose of between 1 and 10 mg per day. In some embodiments, an anti-androgen drug such as enzalutamide or bicalutamide is orally administered. In some embodiments, the enzalutamide or bicalutamide is orally administered to a subject (e.g., an adult human) at a daily dose of approximately 10, 15, 20, 25, 30, 35, 40, 50, 60, 70, 80, 90, 100, 125, 150, 175, 200, 225, 250, 300, 350, 400, 500, or more mg per day. In some embodiments, the enzalutamide or bicalutamide is orally administered to a subject (e.g., an adult human) at a daily dose of between 25 and 250 mg per day. In some embodiments, the docetaxel and the enzalutamide or bicalutamide are orally co-administered. For example, the docetaxel can be co-administered at a daily oral dose of between 25 and 200 mg per day with enzalutamide or bicalutamide at a daily oral dose of between 50 and 200 mg per day.

In some cases, the recitation of a dose “per day” refers to the amount of drug administered each day. In other cases, the “per day” dose refers to the average amount per day of drug administered over a period of time. Thus, if a drug is administered once a week at 100 mg, then the “per day” dose would be approximately equal to (100 mg/7 days=) 14.3 mg per day.

In some cases, the taxane (e.g., docetaxel) is administered once every 1, 2, 3, 4, 5, or 6 weeks, or more. For example, docetaxel, or another taxane, can be administered (e.g., in combination with an inhibitor of docetaxel efflux) every three weeks for 1, 2, 3, 4, 5, 6, 7, 8, 9, 10, or more cycles.

Following successful treatment, it may be desirable to have the subject undergo maintenance therapy to prevent the recurrence of the cancer (e.g., prostate cancer).

Determination of an effective amount is well within the capability of those skilled in the art, especially in light of the detailed disclosure provided herein. Generally, an efficacious or effective amount of an composition is determined by first administering a low dose or small amount of the composition, and then incrementally increasing the administered dose or dosages, adding a second or third medication as needed, until a desired effect of is observed in the treated subject with minimal or no toxic side effects.

Single or multiple administrations of the compositions are administered depending on the dosage and frequency as required and tolerated by the patient. In any event, the composition should provide a sufficient quantity of the compositions of this invention to effectively treat the patient. Generally, the dose is sufficient to treat or ameliorate symptoms or signs of disease without producing unacceptable toxicity to the patient.

### VII. Kits, Containers, Devices, and Systems

A wide variety of kits, systems, and compositions can be prepared according to the present invention, depending upon the intended user of the kit and system and the particular needs of the user. In some embodiments, the present invention provides a kit that includes a taxane (e.g., docetaxel), and/or an inhibitor of taxane efflux, such as an inhibitor of ABCB1 efflux and/or ATPase activity (e.g., an anti-androgen drug such as bicalutamide or enzalutamide), alone or in combination with other compounds.

In some embodiments, the present invention provides a kit that includes a container containing a taxane and a container (e.g., a separate container) containing a drug that reduces efflux of the taxane from the cell. In some cases, the drug that reduces efflux of taxane from the cell is an inhibitor of ABCB1 efflux and/or an inhibitor of ABCB1 ATPase activity. In some cases, the drug that reduces efflux of the taxane is an anti-androgen drug, such as bicalutamide, enzalutamide, or a combination thereof. In some cases, the anti-androgen drug is bicalutamide. In some cases, the anti-androgen drug is enzalutamide. In some cases, the anti-androgen drug contains bicalutamide and enzalutamide.

The taxane in the kit can be any taxane suitable for administration to a subject in need thereof or contacting with a cancer cell. In some cases, the taxane is selected from the group consisting of paclitaxel, docetaxel, cabazitaxel, taxol, hongdoushan A, hongdoushan B, hongdoushan C, baccatin I, baccatin II, a 10-deacetylbaccatin, and derivatives or combinations thereof. In some cases, the taxane is docetaxel.

The compositions of the present invention, including but not limited to compositions containing a taxane, such as docetaxel, and/or drugs that reduce efflux of taxane from a cell, such as bicalutamide or enzalutamide, may, if desired, be presented in a bottle, jar, vial, ampoule, tube, or other container-closure system approved by the Food and Drug Administration (FDA) or other regulatory body, which may provide one or more dosages containing the active ingredient. The package or dispenser may also be accompanied by a notice associated with the container in a form prescribed by a governmental agency regulating the manufacture, use, or sale of pharmaceuticals, or the notice indicating approval by the agency. In certain aspects, the kit may include a formulation or composition as taught herein, a container closure system including the formulation or a dosage unit form including the formulation, and a notice or instructions describing a method of use as taught herein.

In some embodiments, the kit includes a container which is compartmentalized for holding the various elements of a formulation (e.g., the dry ingredients and the liquid ingredients) or composition, instructions for making the formulation or composition, and instructions for preventing, treating, or controlling cancer, e.g., prostate cancer or castrate-resistant prostate cancer, docetaxel-resistant cancer (e.g., prostate cancer), or a combination thereof. In certain embodiments, the kit may include the pharmaceutical preparation in dehydrated or dry form, with instructions for its rehydration (or reconstitution) and administration.

Kits with unit doses of the active composition, e.g. in oral, rectal, transdermal, or injectable doses (e.g., for intramuscular, intravenous, or subcutaneous injection), are provided. In such kits, in addition to the containers containing the unit doses will be an informational package insert describing the use and attendant benefits of the composition in preventing, treating, or controlling cancer, e.g., prostate cancer, castrate-resistant prostate cancer, docetaxel-resistant prostate cancer, or a combination thereof. Suitable active compositions and unit doses are those described herein.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

### EXAMPLES

### Example 1: Anti-Androgens Inhibit ABCB1 Efflux and ATPase Activity and Reverse Docetaxel Resistance in Advanced Prostate Cancer

Introduction

Anti-androgens, such as bicalutamide and enzalutamide, were examined to determine whether they could inhibit ABCB1 activity and overcome resistance to taxanes such as docetaxel.

Materials and Methods

Cell lines and cell culture: DU145 cells were obtained from the American Type Culture Collection (ATCC). All experiments with cell lines were conducted within 6 months of receipt from ATCC or resuscitation after cryopreservation. ATCC uses short tandem repeat (STR) profiling for testing and authentication of cell lines. C4-2B prostate cancer cells were kindly provided and authenticated by Dr. Leland Chung (Cedars-Sinai Medical Center, Los Angeles, Calif.). The cells were cultured in RPMI-1640 medium containing 10% complete fetal bovine serum (FBS) 100 units/mL penicillin and 0.1 mg/mL streptomycin and maintained at 37° C. in a humidified incubator with 5% CO2. Docetaxel-resistant C4-2B-TaxR (TaxR) and DU145-DTXR cells were generated from parental C4-2B and DU145 cells respectively by gradually increasing concentrations of docetaxel in the culture medium as previously described(23). TaxR and DU145-DTXR cells were maintained in 5 nM docetaxel-containing medium.

Materials and Reagents:

Docetaxel (CAS#114977-28-5) was purchased from TSZ CHEM (Framingham, Mass.). Bicalutamide (Cat: #B3209) were purchased from LKT Laboratories, Int. Elacridar (Cat: #143664-11-3) and Rhodamine 123 (Cat: #62667-70-9) were purchased from Sigma-Aldrich. Anti-ser15 phosphorylated p53 antibody was obtained from Cell Signaling Technology, Int. Cleaved PARP, wt-p53 and tubulin antibodies were obtained from Santa Cruz Biotechnologies (Santa Cruz, Calif.).

Preparation of Whole Cell Extracts:

Cells were harvested, rinsed twice with PBS, and lysed in high-salt buffer (10 mM HEPES[pH 7.9], 0.25 M NaCl, 0.1% NP-40) supplemented with protease inhibitors (Roche, Basel, Switzerland). Whole cell extracts were prepared as previously described(29). Protein concentration was determined with Coomassie Plus protein assay kit (Piece, Rockford, Ill.).

Western Blot Analysis:

Equal amounts of total cell lysates (40 μg of protein) were loaded on 10% or 12% SDS-PAGE, and electrophoretically transferred onto nitrocellulose membranes. After blocking in 5% non-fat milk in 1×PBS/0.1% Tween-20 at room temperature for 1 hour, membranes were washed three times with 1×PBS/0.1% Tween-20. The membranes were incubated overnight with primary antibodies at 4° C. Proteins were visualized by enhanced chemiluminescence kit (Millipore) after incubation with the appropriate horseradish peroxidase-conjugated secondary antibodies as previously described(30).

Rhodamine 123 Efflux Assay:

Cells were seeded in 6-well plates at a density of 2×10⁵ cells per well. Cells were treated with elacridar, enzalutamide, and bicalutamide overnight, and then incubated with 1 μM Rhodamine 123 for 4 hours. The cells were then washed three times with PBS. Fluorescent pictures were taken. Each experiment was repeated at least 3 times.

Cell Growth Assay:

Cells were seeded in 12-well plates at a density of 1×10⁵ cells per well. Cells were treated and total cell numbers were counted using a Coulter cell counter.

Cell Death ELISA:

TaxR cells were seeded in 12-well plates at a density of 1×10⁵ cells per well and were treated. DNA fragmentation in the cytoplasmic fraction of cell lysates was determined using the Cell Death Detection ELISA kit (Roche, Indianapolis, Ind.) according to the manufacturer's instructions. Apoptotic cell death was measured at 405 nm absorbance.

Clonogenic Assay:

TaxR cells were treated for 6 hours. Two thousands cells were then plated in 100 mm dishes for 14 days. The cells were fixed with 4% formaldehyde for 10 min, stained with 0.5% crystal violet for 30 minutes, and the numbers of colonies were counted.

ABCB1 ATPase Activity Assay:

ATPase activity was determined by Pgp-Glo™ assay systems (Promega, Madison, Wis.) following the user protocol provided by the manufacturer. Na₃VO₄ was used as an ABCB1 ATPase inhibitor. The activity of ABCB1 ATPase was measured in the presence of test compounds incubated with 25 μM recombinant human ABCB1 membranes at 37° C. for 40 min. Luminescence was initiated by ATP detection buffer and luminescent activity was immediately read on Glomax 96-microplate luminometer (Promega, Madison, Wis.). To rule out the possibility of false positives (unexpected stimulation of ABCB1 ATPase activity by bicalutamide or enzalutamide), the activity of ABCB1 ATPase in cells receiving different concentrations of bicalutamide or enzalutamide plus Na₃VO₄ was compared with treatment with Na₃VO₄ alone. The differences between the average luminescent signals from Na₃VO₄ samples and bicalutamide or enzalutamide plus Na₃VO₄ samples reflect the luciferase inhibitory values (ΔRLU) by different concentrations of bicalutamide or enzalutamide. The ABCB1 ATPase activity was measured in the presence of docetaxel (50 μM) plus different concentrations of bicalutamide or enzalutamide. The average luminescent signals from docetaxel plus bicalutamide or enzalutamide were normalized to the luciferase inhibitory values (ΔRLU) of bicalutamide or enzalutamide. The difference in luminescent signal between Na₃VO₄-treated samples and untreated samples represents the basal ABCB1 ATPase activity. The ABCB1 ATPase activity affected by bicalutamide or enzalutamide was calculated by the difference in luminescent signal between Na₃VO₄-treated samples and the adjusted bicalutamide or enzalutamide-treated samples.

In Vivo Tumorigenesis Assay:

C4-2B, TaxR and DU145-DTXR cells (4×10⁶) were mixed with matrigel (1:1) and injected subcutaneously into the flanks of 6 to 8 week-old male SCID mice. C4-2B derived tumor-bearing mice (tumor volume around 50-100 mm³) were randomized into two groups (with six tumors each group) and treated as follows: (i) vehicle control (5% Tween 80 and 5% ethanol in PBS, i.p.), (ii) docetaxel (10 mg/kg, p.o.). TaxR derived tumor-bearing mice and DU145-DTXR derived tumor-bearing mice (tumor volume around 50-100 mm³) were randomized into four groups (with six tumors each group) and treated as follows: (i) vehicle control (5% Tween 80 and 5% ethanol in PBS, i.p.), (ii) docetaxel (10 mg/kg, i.p., once a week), (iii) bicalutamide (25 mg/kg, p.o., 5 days a week), and (iv) docetaxel (10 mg/kg, i.p., once a week)+bicalutamide (25 mg/kg, p.o., 5 days a week). Tumors were measured using calipers twice a week and tumor volumes were calculated using length×width²/2. Tumor tissues were harvested after 3 weeks of treatment.

Immunohistochemistry:

Tumors were fixed by formalin and paraffin-embedded tissue blocks were dewaxed, rehydrated, and blocked for endogenous peroxidase activity as previously described(31). Antigen retrieving was performed in sodium citrate buffer (0.01 mol/L, pH 6.0) in a microwave oven at 1,000 W for 3 minutes and then at 100 W for 2-minutes. Nonspecific antibody binding was blocked by incubating with 10% FBS in PBS for 30 minutes at room temperature. Slides were then incubated with anti-Ki67 (1:500, NeoMarker) at room temperature for 30 minutes. Slides were then washed and incubated with biotin-conjugated secondary antibodies for 30 minutes, followed by incubation with avidin DH-biotinylated horseradish peroxidase complex for 30 minutes (Vectastain ABC Elite Kit; Vector Laboratories). The sections were developed with the Diaminobenzidine Substrate Kit (Vector Laboratories) and counterstained with hematoxylin. Nuclear stained cells were scored and counted in 3 different areas of the tumor. Images were taken with an Olympus BX51 microscope equipped with DP72 camera.

Statistical Analysis:

All data presented in this example are depicted as mean±SD. Statistical significance between groups was determined by one-way ANOVA followed by the Scheffer procedure for comparison of means. P<0.05 was considered significant.

Results

ABCB1 Efflux Activity is Increased in Docetaxel-Resistant Cells:

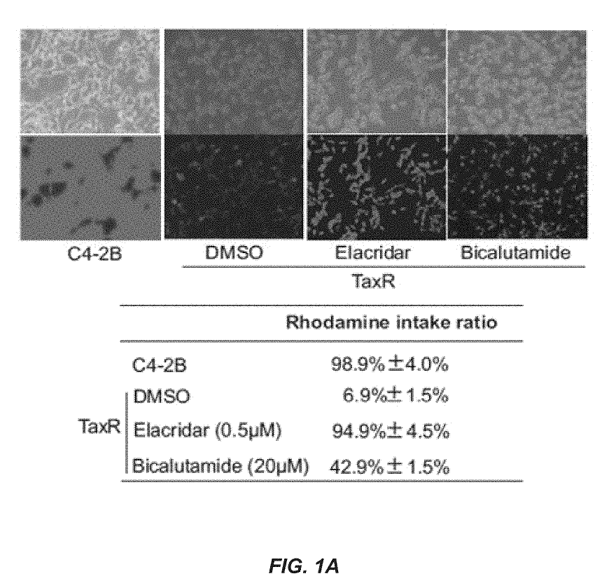

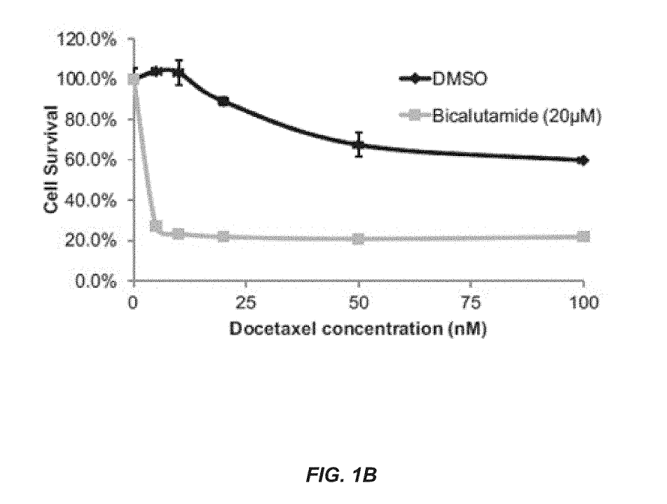

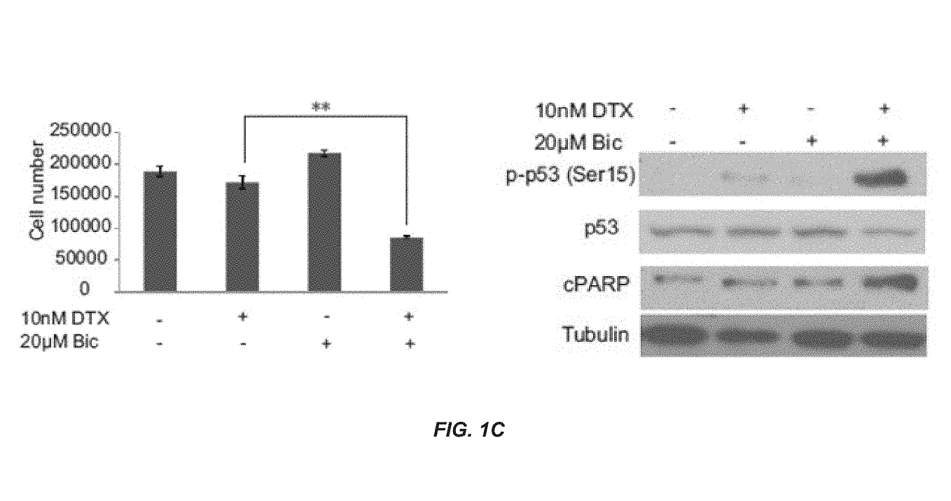

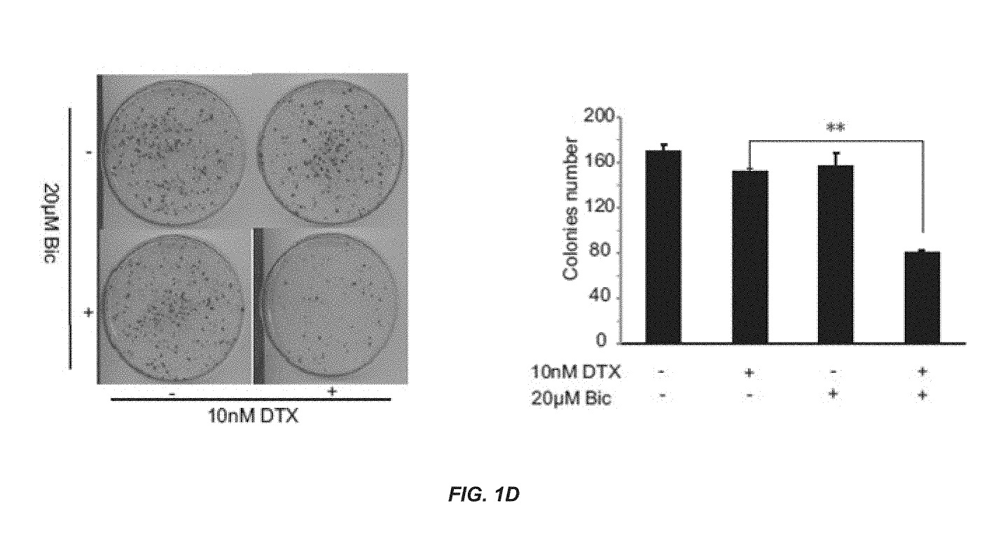

Overexpression of the ABC transporters is known to be involved in multidrug resistance (MDR) in cancer(32). One of the major members of the ABC transporters related to MDR in cancer cells is ABCB1 (P-glycoprotein, ABCB1/MDR1). ABCB1 transports a large number of hydrophobic compounds out of cells including cancer chemotherapeutics agents such as taxanes (like docetaxel,) anthracyclines, and alkaloids (33). We previously demonstrated that ABCB1 is overexpressed in docetaxel-resistant TaxR cells compared to docetaxel sensitive C4-2B parental cells (23). Similarly, others have shown that ABCB1 is also overexpressed in docetaxel-resistant DU145R and CWR22rv1R cells derived from parental DU145 and CWR22rv1 cells, respectively (34). In the present study, we first examined ABCB1 activity in both parental C4-2B cells and ABCB1 overexpressing TaxR cells by using rhodamine efflux assay. Rhodamine 123 is a member of the rhodamine family of fluorescent dyes which is exported by ABCB1 and is routinely used to examine membrane transport by ABCB1(35). Due to the fact that the efflux of rhodamine 123 can be inhibited by other ABCB1 modulators, quantification of rhodamine 123 uptake into cells is an excellent indicator of ABCB1 transport activity, and is very useful in screening for novel ABCB1 inhibitors (36, 37). After 4 hours of incubation in 1 μg/mL rhodamine 123, both parental C4-2B and TaxR cells were washed with 1×PBS three times and the intracellular rhodamine 123 intensities were observed under fluorescent microscope. As shown in FIG. 1A, the intracellular rhodamine accumulation decreased significantly in ABCB1 overexpressing TaxR cells compared to C4-2B cells. TaxR cells had 6.9% rhodamine intake, whereas the rhodamine intake in C4-2B cells was 98.9%. These results suggest that almost all of the docetaxel was taken up into C4-2B parental cells and remained inside of the cells. Conversely, the majority of docetaxel was transported out of the TaxR cells.

Bicalutamide Inhibits the Efflux Activity of ABCB1 and Reverses Docetaxel Resistance in Taxr Cells In Vitro:

It has been reported that combination of androgen deprivation therapy with taxanes could improve therapeutic response of taxanes in CRPC (38). Therefore, we examined whether anti-androgens such as bicalutamide could affect the response of TaxR cells to docetaxel treatment. We first determined if bicalutamide affects the efflux activity of ABCB1 in TaxR cells. A rhodamine efflux assay was performed in TaxR cells treated with bicalutamide or vehicle control. After the cells were incubated with bicalutamide or control for 24 hrs, the rhodamine intake ratio significantly increased from 6.9% to 42.9% (FIG. 1A). This suggests that bicalutamide has the ability to affect ABCB1 efflux activity.

Reversal of ABCB1-mediated multidrug resistance can be achieved by either altering ABCB1 expression or inhibiting ABCB1 efflux activity (39, 40). Since ABCB1 was identified as a critical mechanism leading to docetaxel resistance in TaxR cells (23), we determined whether bicalutamide could restore docetaxel sensitivity by inhibiting ABCB1 efflux activity. In order to analyze the effect of bicalutamide on docetaxel resistance in prostate cancer cells, a cell growth assay was performed in TaxR cells. TaxR cells were treated with increasing concentrations of docetaxel for 24 hrs alone and in combination with 20 μM bicalutamide. As depicted in FIG. 1B, bicalutamide decreased the IC₅₀ value of docetaxel in TaxR cells from 140 nM to ˜5 nM.

To further examine whether bicalutamide restores docetaxel sensitivity of TaxR cells, we treated TaxR cells with 10 nM docetaxel alone or in combination with 20 μM bicalutamide. Treatment with 20 μM bicalutamide had minimal effects on the growth of TaxR cells (FIG. 1C, left panel). However, the combination of 10 nM docetaxel and 20 μM bicalutamide reduced the growth of TaxR cells to ˜40%-50%. The combination treatment led to induction of p53 phosphorylation and cPARP expression (FIG. 1C, right panel). The effect of combination bicalutamide/docetaxel treatment on TaxR cell clonogenic ability was also determined. The combination treatment reduced clone number by ˜40%-50% (FIG. 1D). Taken together, these data suggest that bicalutamide restores docetaxel sensitivity in TaxR cells in vitro.

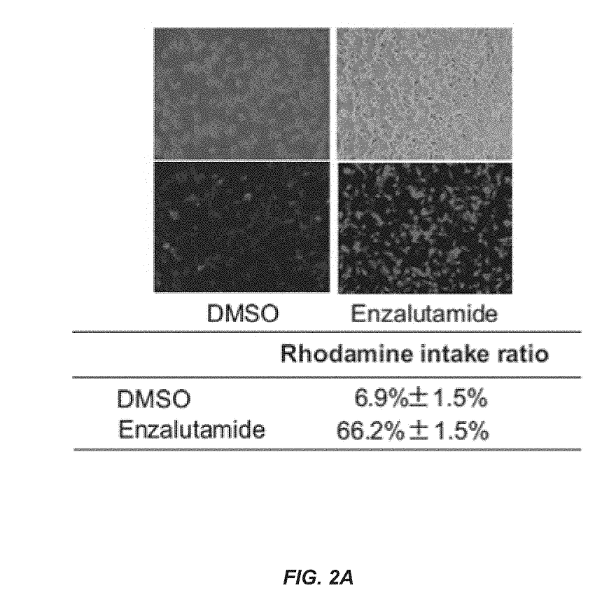

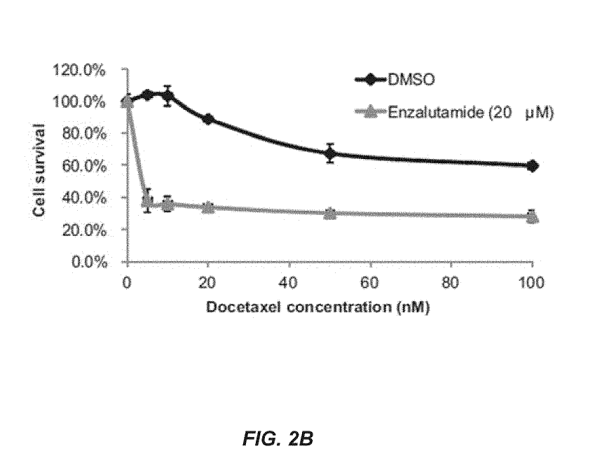

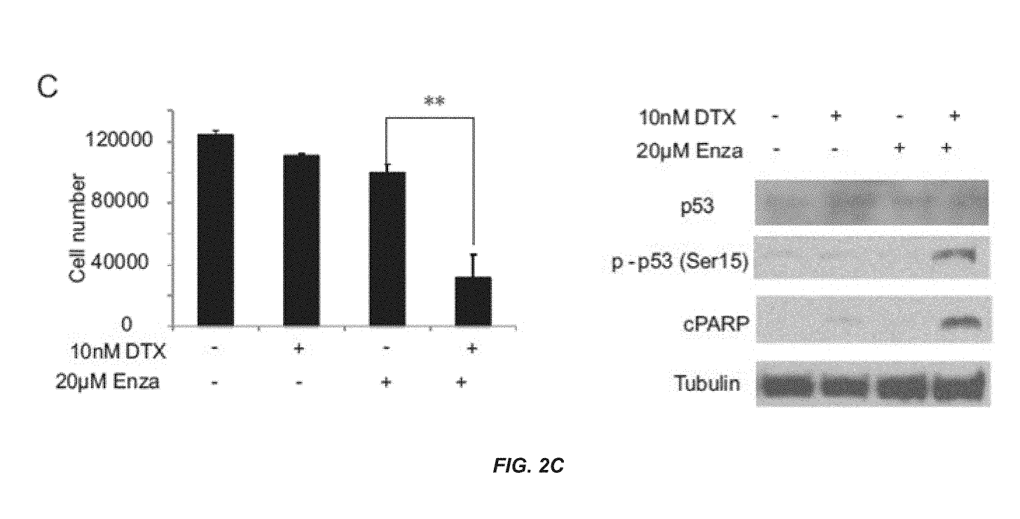

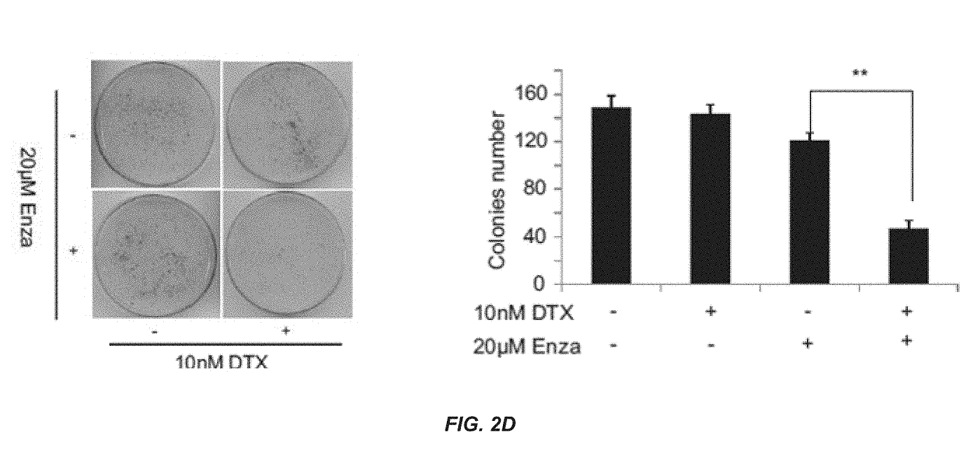

Enzalutamide is a recently FDA-approved drug for patients with CRPC who have failed docetaxel-based chemotherapy as well as for patients with CRPC prior to docetaxel treatment. Since enzalutamide is considered the second generation drug of bicalutamide, we examined if enzalutamide could affect ABCB1 activity similar to bicalutamide. TaxR cells were treated with enzalutamide for 48 hrs and rhodamine efflux activity was determined. TaxR cells had an increased rhodamine intake from 6.9% to 66.2% in the presence of enzalutamide treatment (FIG. 2A), suggesting that enzalutamide could inhibit ABCB1 efflux activity. Next, we examined if enzalutamide could reverse resistance to docetaxel in TaxR cells. TaxR cells were treated with increasing concentrations of docetaxel for 24 hr alone or in combination with 20 μM enzalutamide. As shown in FIG. 2B, 20 μM enzalutamide decreased the IC₅₀ value of docetaxel in TaxR cells from 140 nM to ˜3 nM. Similar to bicalutamide, the combination of 20 μM of enzalutamide with 10 nM docetaxel significantly reduced the growth of TaxR cells (FIG. 2C, left panel) and induced phosphorylation of p53 at Ser15 and the cleavage of PARP (FIG. 2C, right panel). Additionally, combination of docetaxel with enzalutamide reduced clone number by ˜50% in clonogenic assays (FIG. 2D), suggesting that the combination of docetaxel and enzalutamide induces apoptotic cell death.

Anti-Androgens Inhibit the ATPase Activity of ABCB1:

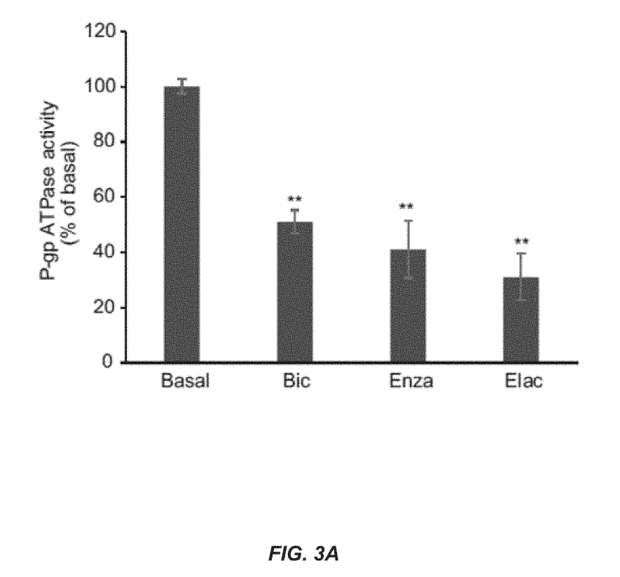

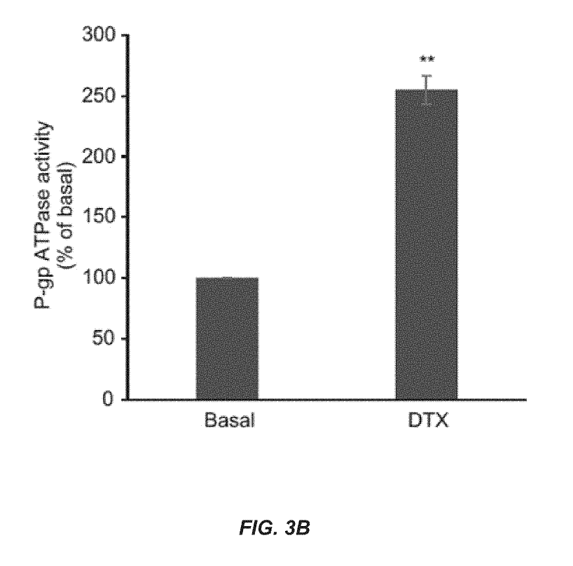

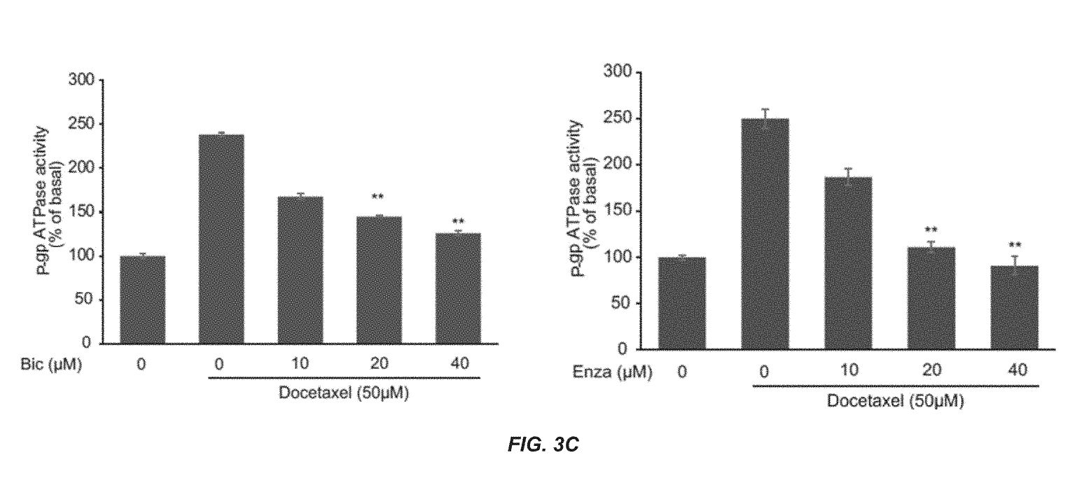

ABCB1, also known as P-glycoprotein, functions as an ATP-dependent drug efflux transporter. Compounds that interact with ABCB1 can either be identified as substrates or inhibitors which stimulate or inhibit the ATPase activity of ABCB1 respectively. In this study, we tested the effects of bicalutamide and enzalutamide on ABCB1 ATPase activity using the Pgp-Glo™ Assay System. As shown in FIG. 3A, the ABCB1 ATPase inhibitor elacridar at 2.5 μM inhibited ABCB1 ATPase activity by 69%. Anti-androgens bicalutamide and enzalutamide decreased ABCB1 ATPase activity in a dose-dependent manner, with 45% and 60% inhibition at concentration of 40 μM respectively, indicating both anti-androgens functioned as ABCB1 ATPase inhibitors. It has been reported that paclitaxel and docetaxel are both substrates for ABCB1-mediated efflux and were able to stimulate ABCB1 ATPase activity (41, 42). In addition, we tested the effect of docetaxel on ABCB1 ATPase activity. As shown in FIG. 3B, 50 μM of docetaxel significantly stimulated ABCB1 ATPase activity by ˜150%. Bicalutamide (FIG. 3C, left panel) and enzalutamide (FIG. 3C, right panel) inhibit the docetaxel-stimulated ABCB1 ATPase activity in a dose-dependent manner. Taken together, these data along with the rhodamine efflux assay illustrate that bicalutamide and enzalutamide can reverse docetaxel resistance in ABCB1-overexpressing cells through inhibiting the ATPase activity of ABCB1.

Bicalutamide Reverses Docetaxel Resistance in Taxr Cells In Vivo:

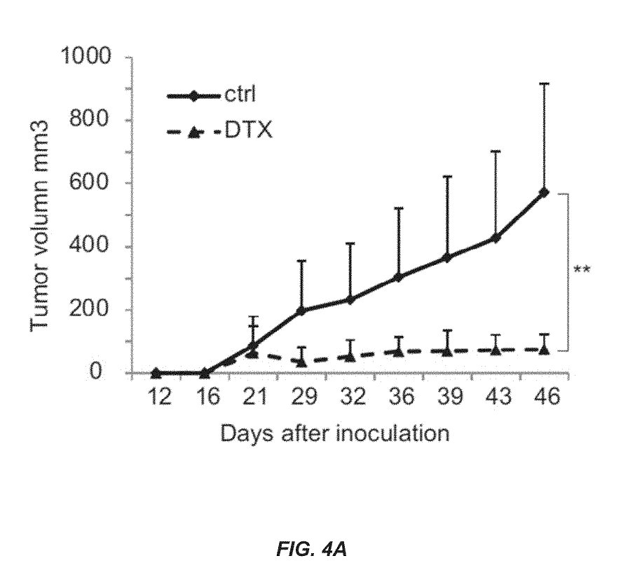

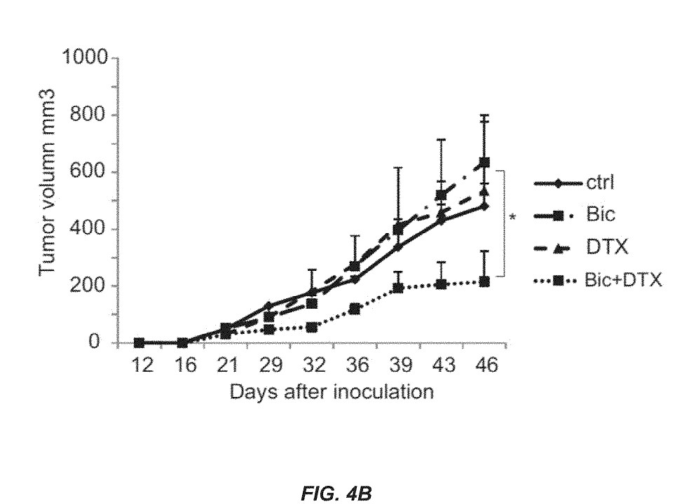

To test if bicalutamide can overcome docetaxel resistance in vivo, docetaxel-resistant TaxR cells and parental C4-2B cells were injected into SCID mice s.c. on the flank. The mice developed tumors three weeks after injection. The mice injected with C4-2B cells were then divided into two groups to receive either vehicle or docetaxel treatments. The mice injected with TaxR cells were divided into four groups to receive either vehicle as controls, docetaxel or bicalutamide alone or with combination treatment. As hypothesized, docetaxel significantly repressed C4-2B tumor growth (FIG. 4A). Neither docetaxel nor bicalutamide treatment alone inhibited tumor growth in TaxR cells, while combinatory treatment with docetaxel and bicalutamide significantly inhibited tumor growth of TaxR cells (FIG. 4B). These results indicate that TaxR cells are resistant to docetaxel, and that combination of bicalutamide with docetaxel overcomes this resistance both in vitro and in vivo.

Bicalutamide Reverses Docetaxel Resistance in DU145-DTXR Cells:

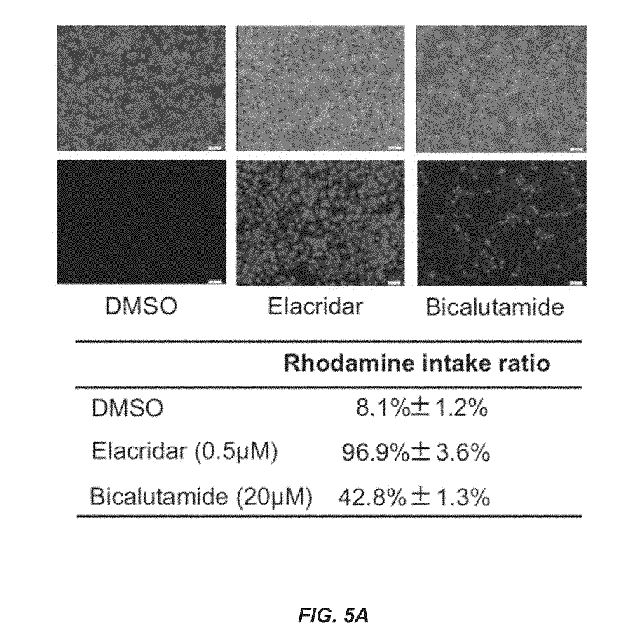

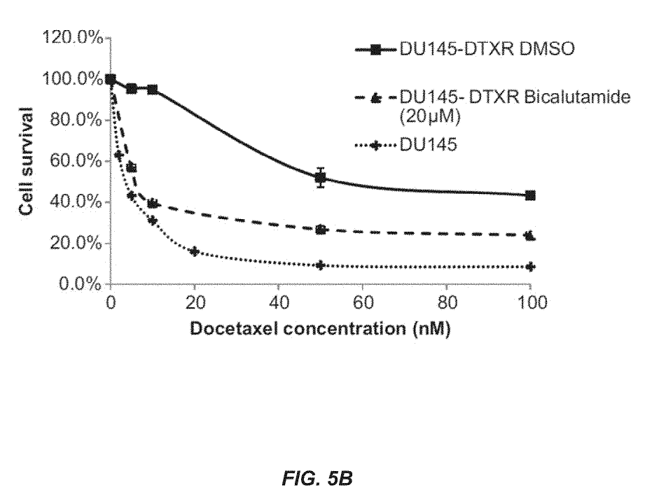

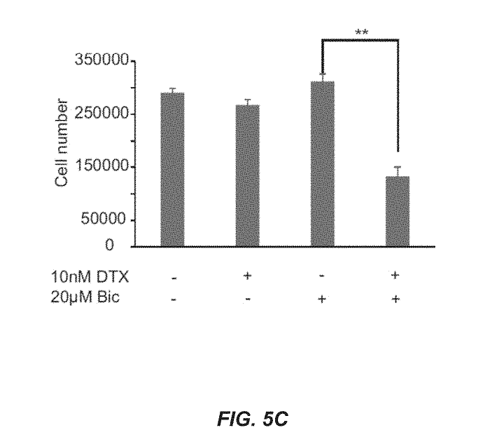

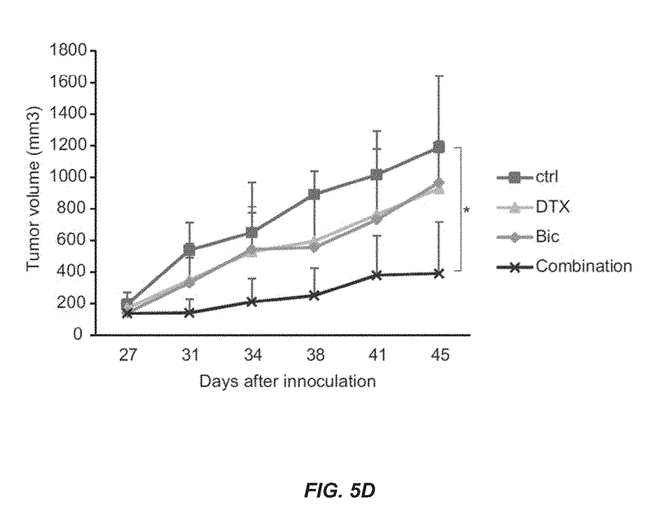

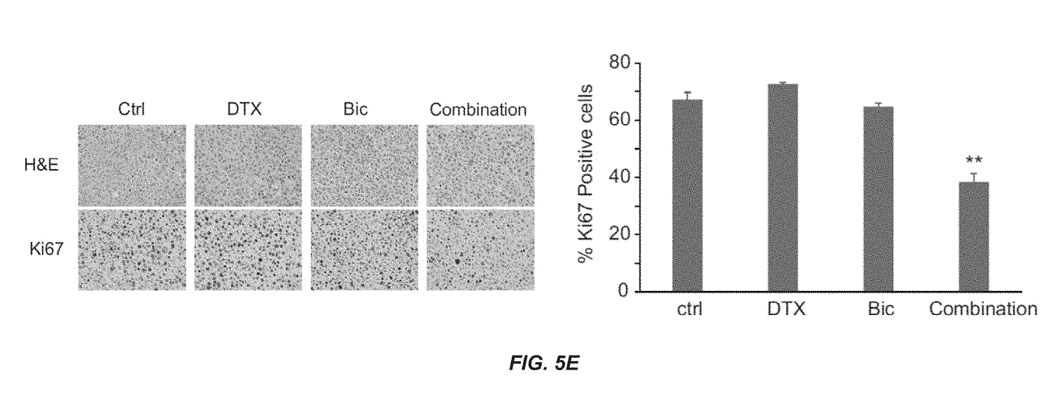

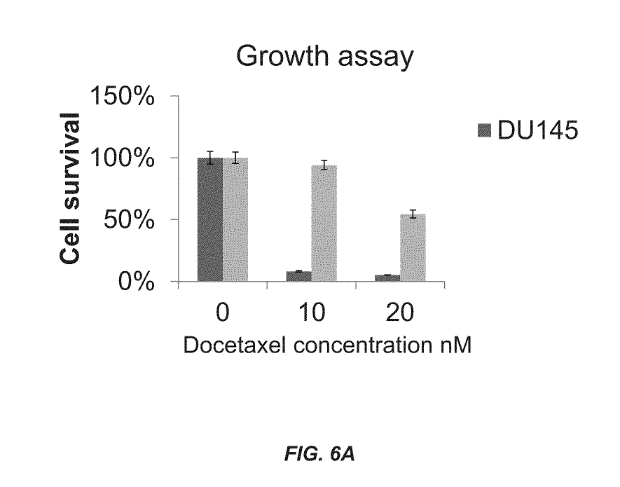

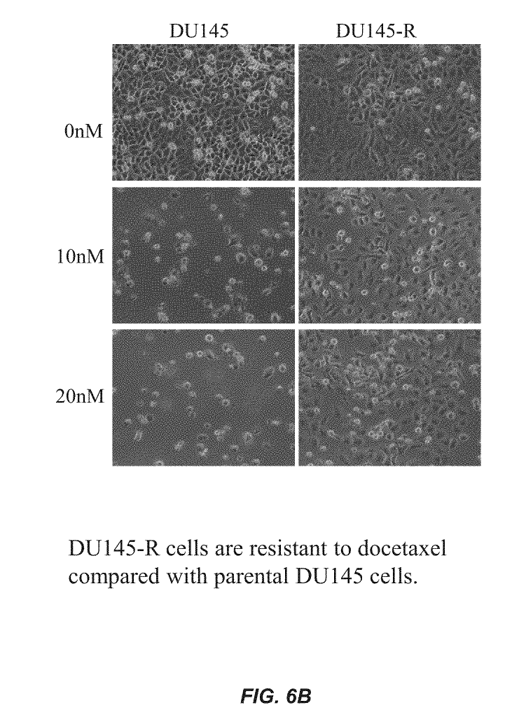

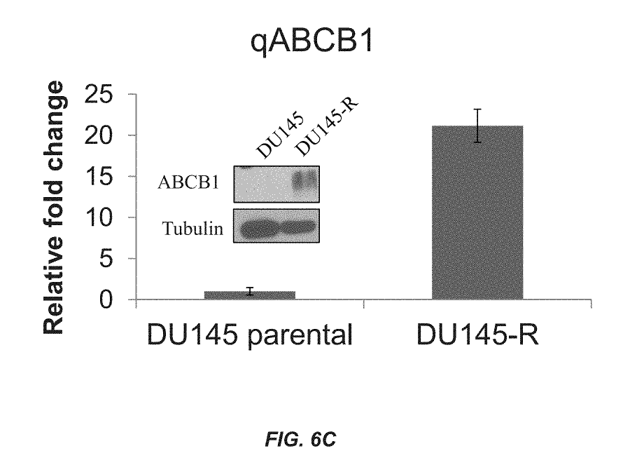

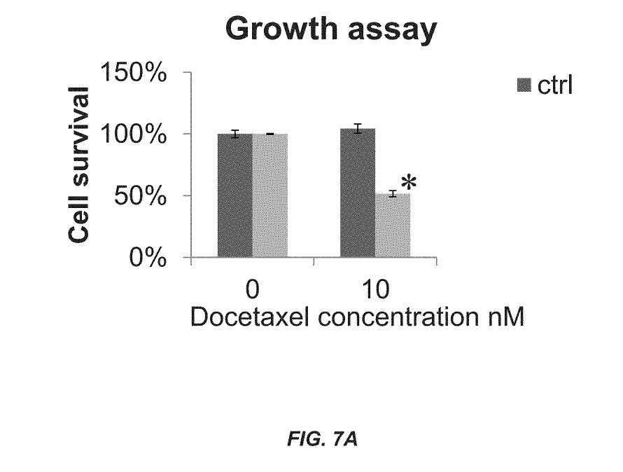

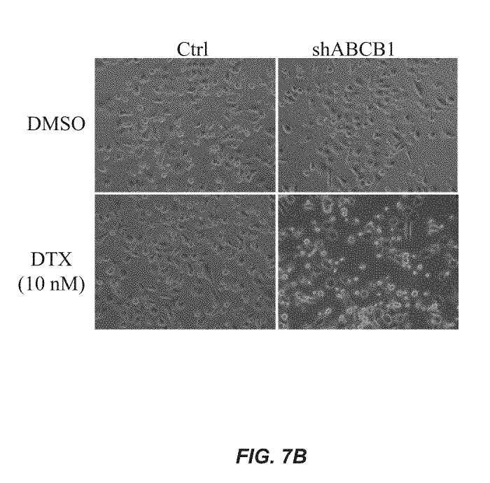

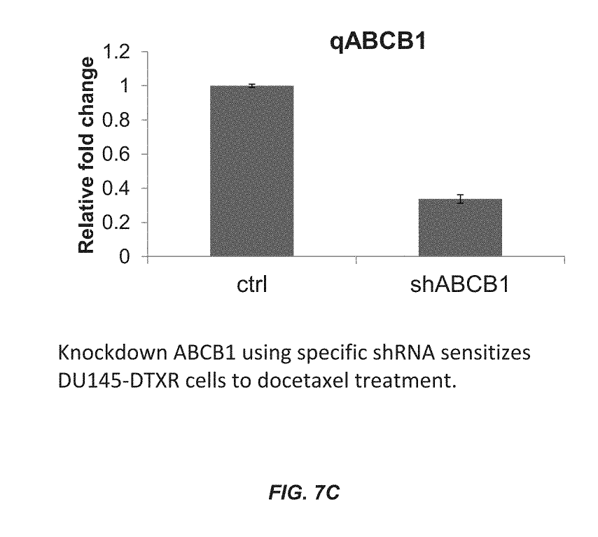

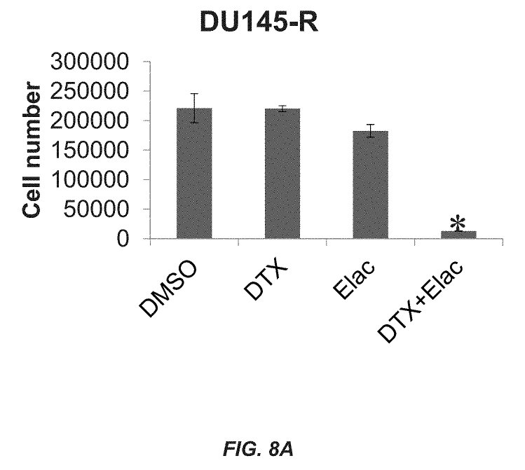

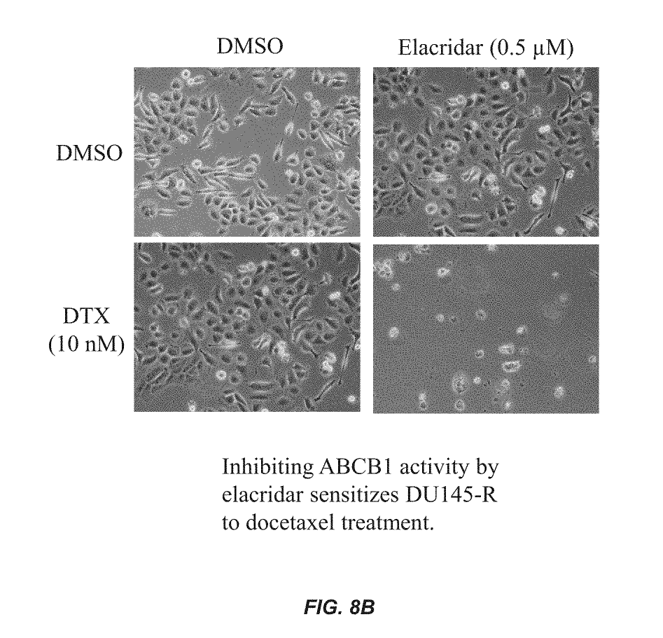

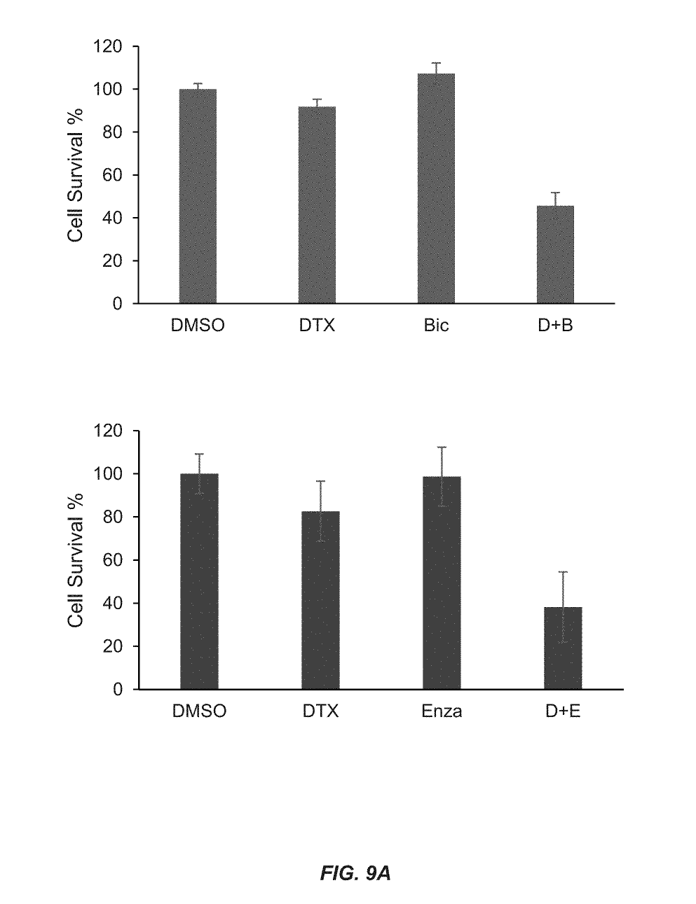

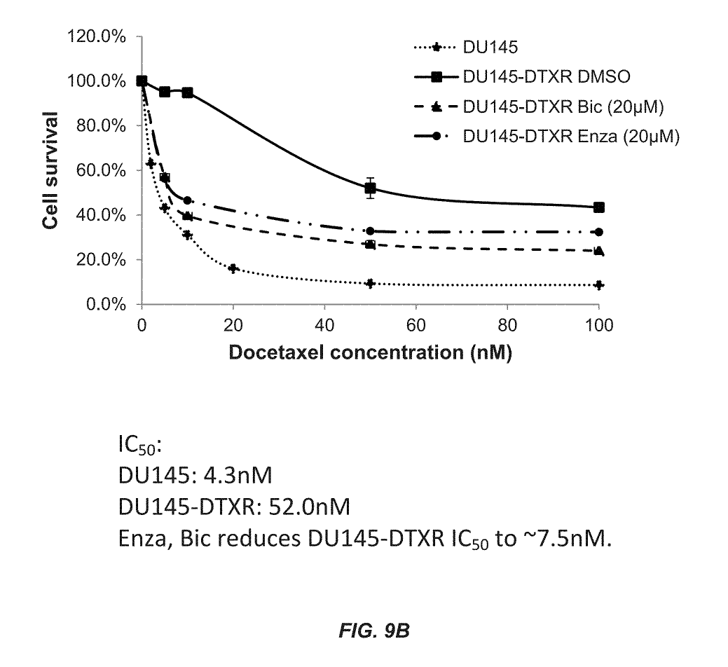

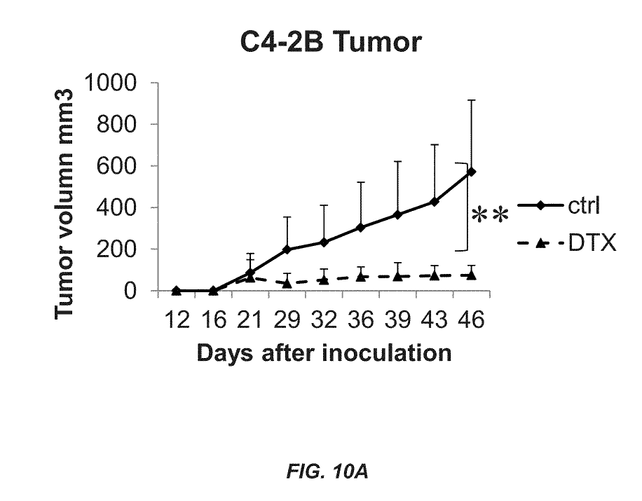

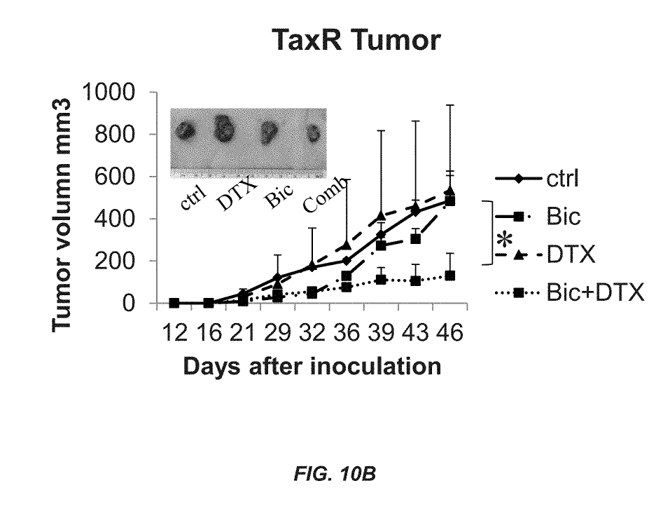

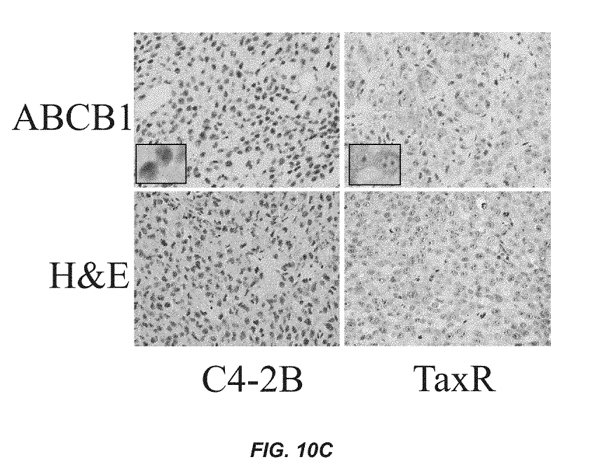

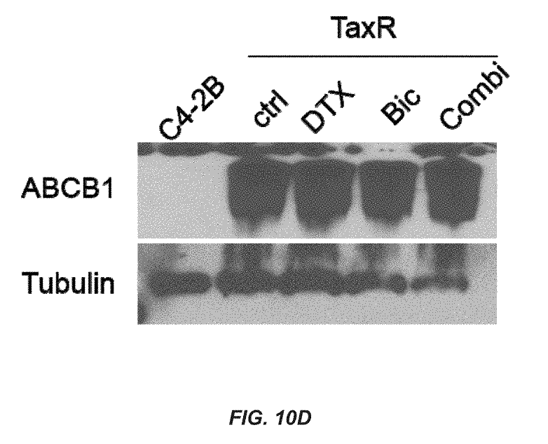

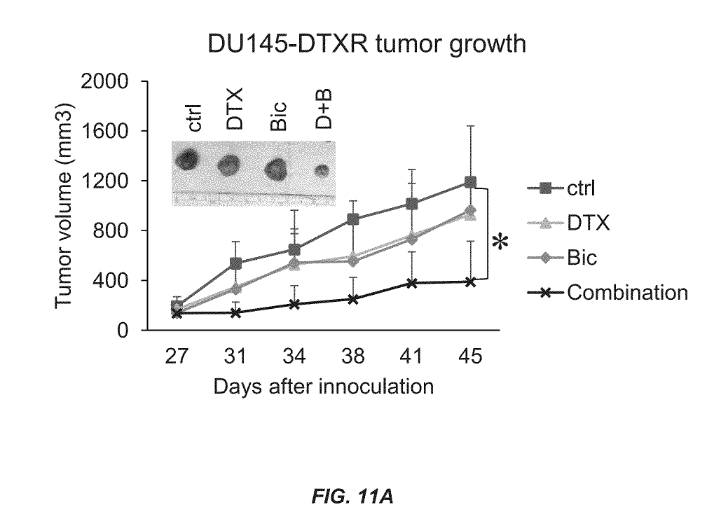

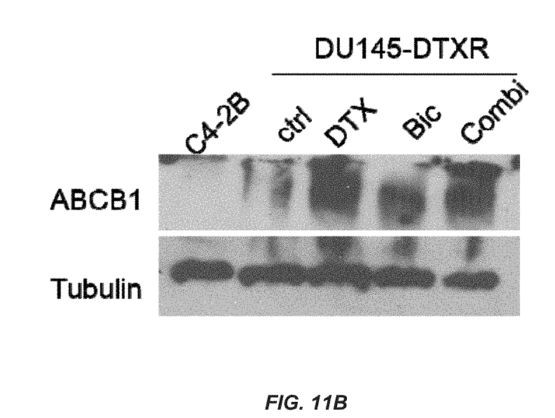

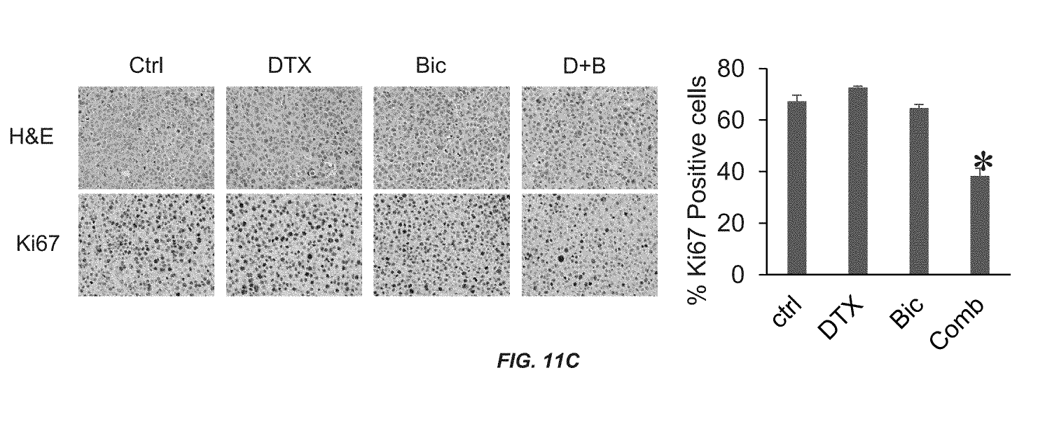

DU145-DTXR cells are another docetaxel-resistant prostate cancer cell line generated from AR-negative parental DU145 cells in our laboratory (23). Our previous data showed that similar to TaxR cells, ABCB1 is overexpressed in DU145-DTXR cells compared to parental DU145 cells (23). These data suggested that overexpression of ABCB1 serves as a general mechanism of docetaxel resistance in prostate cancer. To determine whether the effect of bicalutamide on ABCB1 efflux activity in DU145-DTXR is similar to TaxR cells, rhodamine efflux assay was performed. Bicalutamide increased the rhodamine intake ratio from 8.1% to 42.8% (FIG. 5A). The IC₅₀ to docetaxel in DU145-DTXR cells is 52.0 nM compared with 4.3 nM in parental DU145 cells (FIG. 5B). Further study has shown that 20 μM bicalutamide decreased the IC₅₀ value of docetaxel in DU145-DTXR cells from 52.0 nM to ˜7.5 nM (FIG. 5B). To test the effects of bicalutamide on docetaxel resistance of DU145-DTXR cells, we treated DU145-DTXR cells with 10 nM docetaxel alone or in combination with 20 μM bicalutamide. Although docetaxel or bicalutamide alone had only minimal effects on cell growth, combining docetaxel and bicalutamide significantly inhibited the growth of DU145-DTXR cells (FIG. 5C). These effects were further verified in vivo. Four million DU145-DTXR cells were injected under the flank of 4-6 week old male SCID mice. The mice were randomly divided into four groups when the tumor size reached 50-100 mm³ to receive vehicle control, docetaxel or bicalutamide alone or in a combined treatment. The combination group had a significantly slower tumor growth rate and resulted in much smaller tumors compared with the other three treatment groups (FIG. 5D). Representative tumor samples were analyzed by IHC for Ki67 expression. Combination treatment further decreased Ki67 expression (FIG. 5E). Altogether, these data indicate that bicalutamide reversed ABCB1-mediated docetaxel resistance in DU145-DTXR cells in vitro and in vivo. The data presented in FIGS. 6A-11C further support a finding of docetaxel resistance reversal in taxane-resistant cells by contact with an ABCB1 efflux inhibitor such as elacridar, enzalutamide, bicalutamide, or a combination thereof.

Discussion

Docetaxel has been the first-line treatment for metastatic CRPC since 2004. Acquired resistance to docetaxel treatment is one of the major concerns regarding prostate cancer therapy(43). Previous studies from our laboratory have observed that ABCB1 is up-regulated in docetaxel-resistant TaxR prostate cancer cells and that knockdown of ABCB1 expression sensitized resistant TaxR cells to docetaxel(23). In the current study, we demonstrate that ABCB1 efflux activity in TaxR cells is significantly increased compared to parental C4-2B cells. Anti-androgens such as enzalutamide and bicalutamide inhibit ABCB1 efflux activity and reverse the ABCB1-mediated docetaxel resistance in prostate cancer. Furthermore, combination of bicalutamide or enzalutamide with docetaxel significantly enhances the cytotoxicity of docetaxel independent of AR status.

Overexpression of ABCB1 is one of the main mechanisms leading to docetaxel resistance in CRPC. ABCB1 activity in cultured cells has been assessed by monitoring the efflux of several small, fluorescent molecules such as rhodamine 123 from cells (35). Efflux of rhodamine 123 was reduced when TaxR cells were treated with either bicalutamide or enzalutamide. Bicalutamide inhibited ABCB1 efflux activity by 40% and decreased the IC₅₀ of docetaxel in TaxR cells from 140 nM to around 5 nM, while enzalutamide inhibited ABCB1 efflux activity by ˜60%. Reversal of ABCB1-mediated docetaxel resistance can be achieved by altering ABCB1 expression. The ability of blockage of ABCB1 efflux activity by bicalutamide and enzalutamide is through inhibition of ABCB1 ATPase activity. Both bicalutamide and enzalutamide act as anti-androgens to treat AR-positive prostate cancer. Our finding that bicalutamide/enzalutamide block ABCB1 efflux activity via inhibition of ABCB1 ATPase activity provides a new mechanism of action of these drugs. This novel mechanism of action provides a scientific rational for a combination treatment strategy of bicalutamide/enzalutamide with docetaxel for advanced prostate cancer independent of the status of androgen receptor.

For patients with progressive CRPC previously treated with docetaxel, enzalutamide confers a statistically significant improvement in overall survival, leading to its recent US FDA approval. Enzalutamide impairs AR signaling by inhibiting AR nuclear translocation and DNA binding (31). Clinical studies indicate that targeting AR signaling remains an important therapeutic strategy in docetaxel-resistant CRPC. In this study, we showed a significant growth inhibition effect of enzalutamide on TaxR cells when combined with docetaxel through a reduction of ABCB1 efflux activity. Combination of enzalutamide and docetaxel inhibited cell growth and clonogenic ability accompanied with increased cellular apoptosis of TaxR cells. These findings suggest an additional mechanism of action by enzalutamide in CRPC through inhibition of ABCB1 efflux activity.

Similar to enzalutamide, the previous generation of anti-androgen, bicalutamide, was also found to inhibit ABCB1 efflux activity and re-sensitize docetaxel-resistant cells to docetaxel treatment. Clinically, patients with advanced prostate cancer were initially treated with gonadotropin releasing hormone (GnRH) analogs (which suppress active androgens) alone or in combination with anti-androgens such as bicalutamide. Launched in 1995, bicalutamide is one of the first-generation pure anti-androgens binding to and inhibiting the androgen receptor and is widely used to treat prostate cancer in patients who fail androgen deprivation therapy. Despite the initial response to bicalutamide treatment, almost all of the patients develop CRPC and no longer respond to the drug. Docetaxel is the standard first-line treatment for CRPC. However, relapse eventually occurs due to the development of resistance to docetaxel. Enzalutamide and abiraterone are recently approved therapies for CRPC after the cancer fails to respond to docetaxel. Based on the fact that bicalutamide inhibited ABCB1 efflux activity and reversed the resistance to docetaxel similar to enzalutamide, bicalutamide could be developed as a combination therapy with docetaxel to effectively treat docetaxel-resistant CRPC. Although both abiraterone and enzalutamide have significant benefit for CRPC patients who fail docetaxel-based chemotherapy, these treatments are expensive. Compared to enzalutamide or abiraterone treatment, combination treatment of bicalutamide with docetaxel is more cost effective, and could be developed to treat patients with CRPC who fail docetaxel therapy.

In summary, we identified a novel mechanism of action of the anti-androgens such as bicalutamide and enzalutamide as inhibitors for ABCB1 efflux and ATPase activity. Bicalutamide and enzalutamide desensitize docetaxel-resistant prostate cancer cells to docetaxel treatment. Our studies suggest for the first time that bicalutamide and enzalutamide reverses docetaxel resistance in vitro and in vivo by inhibition of ABCB1 efflux activity, and may be developed as a combination therapy with docetaxel as an effective regiment to treat advanced CRPC independent of AR status.

### REFERENCES

1. Trapman J, Cleutjens K B. Androgen-regulated gene expression in prostate cancer. Semin Cancer Biol. 1997; 8:29-36.
2. Dutt S S, Gao A C. Molecular mechanisms of castration-resistant prostate cancer progression. Future Oncology. 2009; 5:1403-13.
3. Harris W P, Mostaghel E A, Nelson P S, Montgomery B. Androgen deprivation therapy: progress in understanding mechanisms of resistance and optimizing androgen depletion. Nat Clin Pract Urol. 2009; 6:76-85.
4. Fitzpatrick J M, de Wit R. Taxane Mechanisms of Action: Potential Implications for Treatment Sequencing in Metastatic Castration-resistant Prostate Cancer. European Urology.
5. Herbst R S, Khuri F R. Mode of action of docetaxel—a basis for combination with novel anticancer agents. Cancer Treatment Reviews. 2003; 29:407-15.
6. Kim S J, Kim S I. Current treatment strategies for castration-resistant prostate cancer.

Korean J Urol. 2011; 52:157-65.
7. Galletti E, Magnani M, Renzulli M L, Botta M. Paclitaxel And Docetaxel Resistance: Molecular Mechanisms and Development of New Generation Taxanes. ChemMedChem. 2007; 2:920-42.
8. de Bono J S, Logothetis C J, Molina A, Fizazi K, North S, Chu L, et al. Abiraterone and Increased Survival in Metastatic Prostate Cancer. New England Journal of Medicine. 2011; 364:1995-2005.
9. Scher H I, Fizazi K, Saad F, Taplin M-E, Sternberg C N, Miller K, et al. Increased Survival with Enzalutamide in Prostate Cancer after Chemotherapy. New England Journal of Medicine. 2012; 367:1187-97.
10. Wilson L, Tang J, Zhong L, Balani G, Gipson G, Xiang P, et al. New therapeutic options in metastatic castration-resistant prostate cancer: Can cost-effectiveness analysis help in treatment decisions? Journal of Oncology Pharmacy Practice. 2013.
11. Shalli K, Brown I, Heys S D, Schofield A C. Alterations of β-tubulin isotypes in breast cancer cells resistant to docetaxel. The FASEB Journal. 2005.
12. Berrieman H K, Lind M J, Cawkwell L. Do β-tubulin mutations have a role in resistance to chemotherapy? The Lancet Oncology. 2004; 5:158-64.
13. Hari M, Loganzo F, Annable T, Tan X, Musto S, Morilla D B, et al. Paclitaxel-resistant cells have a mutation in the paclitaxel-binding region of β-tubulin (Asp26Glu) and less stable microtubules. Molecular Cancer Therapeutics. 2006; 5:270-8.
14. Hara T, Ushio K, Nishiwaki M, Kouno J, Araki H, Hikichi Y, et al. A mutation in β-tubulin and a sustained dependence on androgen receptor signalling in a newly established docetaxel-resistant prostate cancer cell line. CellBioInt. 2010; 34:177-84.
15. Seruga B, Ocana A, Tannock I F. Drug resistance in metastatic castration-resistant prostate cancer. Nat Rev Clin Oncol. 2011; 8:12-23.
16. Yoshino T, Shiina H, Urakami S, Kikuno N, Yoneda Y, Shigeno K, et al. Bcl-2 Expression as a Predictive Marker of Hormone-Refractory Prostate Cancer Treated with Taxane-Based Chemotherapy. Clin Cancer Res. 2006; 12:6116-24.
17. Sowery R D, Hadaschik B A, So A I, Zoubeidi A, Fazli L, Hurtado-Coll A, et al. Clusterin knockdown using the antisense oligonucleotide OGX-011 re-sensitizes docetaxel-refractory prostate cancer PC-3 cells to chemotherapy. BJU International. 2008; 102:389-97.
18. Zhong B, Sallman D A, Gilvary D L, Pernazza D, Sahakian E, Fritz D, et al. Induction of clusterin by AKT-Role in cytoprotection against docetaxel in prostate tumor cells. Mol Cancer Ther. 2010; 9:1831-41.
19. Kosaka T, Miyajima A, Shirotake S, Suzuki E, Kikuchi E, Oya M. Long-Term Androgen Ablation and Docetaxel Up-Regulate Phosphorylated Akt in Castration Resistant Prostate Cancer. The Journal of Urology. 2011; 185:2376-81.
20. Liu C, Zhu Y, Lou W, Nadiminty N, Chen X, Zhou Q, et al. Functional p53 determines docetaxel sensitivity in prostate cancer cells. Prostate. 2013; 73:418-27.
21. Domingo-Domenech J, Vidal S J, Rodriguez-Bravo V, Castillo-Martin M, Quinn S A, Rodriguez-Barrueco R, et al. Suppression of acquired docetaxel resistance in prostate cancer through depletion of notch- and hedgehog-dependent tumor-initiating cells. Cancer Cell. 2012; 22:373-88.
22. Vidal Samuel J, Rodriguez-Bravo V, Quinn S A, Rodriguez-Barrueco R, Lujambio A, Williams E, et al. A Targetable GATA2-IGF2 Axis Confers Aggressiveness in Lethal Prostate Cancer. Cancer Cell. 2015; 27:223-39.
23. Zhu Y, Liu C, Nadiminty N, Lou W, Tummala R, Evans C P, et al. Inhibition of ABCB1 Expression Overcomes Acquired Docetaxel Resistance in Prostate Cancer. Molecular Cancer Therapeutics. 2013; 12:1829-36.
24. Sharom F J. ABC multidrug transporters: structure, function and role in chemoresistance. Pharmacogenomics. 2008; 9.
25. Amanda J O'Neill M P, Catherine Dowling, Yue Fan, Laoighse Mulrane, William m Gallagher, Darran O'Connor, Robert O'Connor, Aoife Devery, Claire Corcoran, Sweta Rani, Lorraine O'Driscoll, John M Fitzpatrick, R William G Watson. Characterisation and manipulation of docetaxel-resistant prostate cancer cell lines. Molecular Cancer. 2011; 10.
26. Gergely Szakacs J K P, Joseph A. Ludwig, Catherine Booth-Genthe, Michael M. Gottesman. Targeting multidrug resistance in cancer. Nature reviews of drug discovery. 2006; 5:219-34.
27. Catherine Sanchez A M, Hector R. Contreras, Patricia Mendoza, Juan Cabezas, Cristian Acevedo, Christian Huidobro, Enrique A. Castellon Chemotherapy sensitivity recovery of prostate cancer cells by functional inhibition and knock down of multidrug resistance proteins. The Prostate. 2011; 71:1810-7.
28. G. Bhangal BS, S. Halford, M.A., M.R.C.P., J. Wang, B.Sc., M.R.C.P., R. Roylance, B.Sc., M.R.C.P., R. Shah, M.R.C.P., J. Waxman, M.D., F.R.C.P., B.Sc. Expression of the multidrug resistance gene in human prostate cancer. Urologic Oncology. 2000; 5:118-21.
29. Zhu Y, Liu C, Cui Y, Nadiminty N, Lou W, Gao A C. Interleukin-6 induces neuroendocrine differentiation (NED) through suppression of RE-1 silencing transcription factor (REST). The Prostate. 2014; 74:1086-94.
30. Zhu Y, Tummala R, Liu C, Nadiminty N, Lou W, Evans C P, et al. RhoGDIa suppresses growth and survival of prostate cancer cells. The Prostate. 2012; 72:392-8.
31. Liu C, Lou W, Zhu Y, Nadiminty N, Schwartz C T, Evans C P, et al. Niclosamide Inhibits Androgen Receptor Variants Expression and Overcomes Enzalutamide Resistance in Castration-resistant Prostate Cancer. Clinical Cancer Research. 2014; 20:3198-210.
32. Sharom F J. ABC multidrug transporters: structure, function and role in chemoresistance. Pharmacogenomics. 2007; 9:105-27.
33. Shi Z, Tiwari A K, Shukla S, Robey R W, Singh S, Kim I-W, et al. Sildenafil Reverses ABCB1- and ABCG2-Mediated Chemotherapeutic Drug Resistance. Cancer Research. 2011; 71:3029-41.
34. Domingo-Domenech J, Vidal S J, Rodriguez-Bravo V, Castillo-Martin M, Quinn S A, Rodriguez-Barrueco R, et al. Suppression of acquired docetaxel resistance in prostate cancer through depletion of notch- and hedgehog-dependent tumor-initiating cells. Cancer Cell. 2012; 22:373-88.
35. Kimchi-Sarfaty C, Oh J M, Kim I-W, Sauna Z E, Calcagno A M, Ambudkar S V, et al. A “Silent” Polymorphism in the MDR1 Gene Changes Substrate Specificity. Science. 2007; 315:525-8.
36. Sheu M-J, Teng Y-N, Chen Y-Y, Hung C-C. The Functional Influences of Common <italic>ABCB1<italic>Genetic Variants on the Inhibition of P-glycoprotein by <italic>Antrodia cinnamomea</italic>Extracts. PLoS ONE. 2014; 9:e89622.
37. Caceres G, Robey R W, Sokol L, McGraw K L, Clark J, Lawrence N J, et al. HG-829 Is a Potent Noncompetitive Inhibitor of the ATP-Binding Cassette Multidrug Resistance Transporter ABCB1. Cancer Research. 2012; 72:4204-13.
38. Zhu M L, Horbinski C M, Garzotto M, Qian D Z, Beer T M, Kyprianou N. Tubulin-targeting chemotherapy impairs androgen receptor activity in prostate cancer. Cancer Res. 2010; 70:7992-8002.
39. Sanchez C, Mercado A, Contreras H R, Mendoza P, Cabezas J, Acevedo C, et al. Chemotherapy sensitivity recovery of prostate cancer cells by functional inhibition and knock down of multidrug resistance proteins. Prostate. 2011; 71:1810-7.
40. Szakács G, Paterson J K, Ludwig J A, Booth-Genthe C, Gottesman M. Targeting multidrug resistance in cancer. Nat Rev Drug Discov. 2006; 5:219-34.
41. Malofeeva E V, Domanitskaya N, Gudima M, Hopper-Borge E A. Modulation of the ATPase and transport activities of broad-acting multidrug resistance factor ABCC10 (MRP7). Cancer Res. 2012; 72:6457-67.
42. Brooks T A, Minderman H, O'Loughlin K L, Pera P, Ojima I, Baer M R, et al. Taxane-based reversal agents modulate drug resistance mediated by P-glycoprotein, multidrug resistance protein, and breast cancer resistance protein. Mol Cancer Ther. 2003; 2:1195-205.
43. Huang X, Chau C, Figg W. Challenges to improved therapeutics for metastatic castrate resistant prostate cancer: from recent successes and failures. Journal of Hematology & Oncology. 2012; 5:35.

Although the forgoing invention has been described in some detail by way of illustration and example for clarity and understanding, it will be readily apparent to one of ordinary skill in the art in light of the teachings of this invention that certain variations, changes, modifications and substitutions of equivalents may be made thereto without necessarily departing from the spirit and scope of this invention. As a result, the embodiments described herein are subject to various modifications, changes and the like, with the scope of this invention being determined solely by reference to the claims appended hereto. Those of skill in the art will readily recognize a variety of non-critical parameters that could be changed, altered or modified to yield essentially similar results. It is also to be understood that the terminology used herein is for the purpose of describing particular embodiments only, and is not intended to be limiting, since the scope of the present invention will be limited only by the appended claims. In addition, each reference provided herein is incorporated by reference in its entirety to the same extent as if each reference was individually incorporated by reference. Where a conflict exists between the instant application and a reference provided herein, the instant application shall dominate.

1. A composition comprising a taxane and a drug that reduces efflux of the taxane from a cell.

2. The composition of claim 1, wherein the drug that reduces efflux of the taxane comprises an anti-androgen drug.

3. The composition of claim 2, wherein the anti-androgen drug is selected from the group consisting of enzalutamide, bicalutamide, and combinations thereof.

4. The composition of claim 2, wherein the anti-androgen drug is an inhibitor of ABCB1 efflux activity.

5. The composition of claim 2, wherein the anti-androgen drug is an inhibitor of ABCB1 ATPase activity.

6. The composition of claim 1, wherein the taxane is selected from the group consisting of paclitaxel, docetaxel, cabazitaxel, taxol, hongdoushan A, hongdoushan B, hongdoushan C, baccatin I, baccatin II, a 10-deacetylbaccatin, and combinations thereof.

7. The composition of claim 1, wherein the composition comprises docetaxel and enzalutamide or bicalutamide, or the composition comprises docetaxel, enzalutamide, and bicalutamide.

8. The composition of claim 1, wherein the composition is an effective inhibitor of cancer cell proliferation.

9. The composition of claim 8, wherein the composition is an effective inhibitor of proliferation of a cancer cell selected from the group consisting of a prostate cancer cell, a metastatic prostate cancer cell, a castrate-resistant prostate cancer cell, a castration recurrent prostate cancer cell, an androgen insensitive prostate cancer cell, a hormone-resistant prostate cancer cell, and a metastatic castrate-resistant prostate cancer cell.

10. The composition of claim 8, wherein the composition is an effective inhibitor of proliferation of a taxane-resistant cancer cell, preferably a docetaxel-resistant cancer cell.

11. A method for treating cancer comprising administering an effective amount of claim 1.

12. The method of claim 11, wherein the cancer comprises taxane-resistant cancer.

13. The method of claim 12, wherein the taxane-resistant cancer is docetaxel resistant cancer.

14. The method of claim 11, wherein the cancer is prostate cancer.

15. The method of claim 14, wherein the prostate cancer is selected from the group consisting of metastatic prostate cancer, castrate-resistant prostate cancer, castration recurrent prostate cancer, androgen insensitive prostate cancer, hormone-resistant prostate cancer, taxane-resistant prostate cancer, docetaxel-resistant prostate cancer, and metastatic castrate-resistant prostate cancer.

16. A method for reducing or reversing resistance of a taxane-resistant cell to a taxane, or re-sensitizing a taxane-resistant cell to a taxane, the method comprising contacting the cell with an effective amount of an anti-androgen drug selected from the group consisting of enzalutamide, bicalutamide, or a combination thereof, or contacting the cell with an effective amount of a composition of claim 1.

17. The method of claim 16, wherein the cell is docetaxel-resistant, and the method comprises reducing or reversing docetaxel resistance of the docetaxel-resistant cell or re-sensitizing the docetaxel-resistant cell to docetaxel.

18. The method of claim 16, wherein the cell is a cancer cell or a prostate cancer cell.

19. The method of claim 18, wherein the prostate cancer cell is selected from the group consisting of metastatic prostate cancer, castrate-resistant prostate cancer, castration recurrent prostate cancer, androgen insensitive prostate cancer, hormone-resistant prostate cancer, and metastatic castrate-resistant prostate cancer.

20. A method for treating cancer in a subject in need thereof, the method comprising:
simultaneously or sequentially administering to the subject a taxane, and an inhibitor of taxane efflux, wherein the inhibitor of taxane efflux comprises an anti-androgen drug.

21. The method of claim 20, wherein the anti-androgen drug comprises bicalutamide or enzalutamide.

22. The method of claim 20, wherein the method further comprises simultaneously or sequentially administering to the subject a second inhibitor of taxane efflux.

23. The method of claim 22, wherein the method comprises simultaneously or sequentially administering the taxane, bicalutamide, and enzalutamide.

24. The method of claim 20, wherein the cancer is a taxane resistant cancer.

25. A method for inhibiting ABCB1 efflux in a cell comprising contacting the cell with an effective amount of an anti-androgen drug.

26. The method of claim 25, wherein the anti-androgen drug is an inhibitor of ABCB1 ATPase activity.

27. The method of claim 25, wherein the anti-androgen drug is bicalutamide, enzalutamide, or a combination thereof.

28. The method of claim 25, wherein the cell is a cancer cell.

29. The method of claim 28, wherein the cancer cell is a taxane-resistant cancer cell.

30. The method of claim 29, wherein the taxane-resistant cancer cell is docetaxel-resistant.

31. A kit comprising a container containing a taxane and a container containing a drug that reduces efflux of the taxane from a cell.

32. The kit of claim 31, wherein the drug that reduces efflux is an inhibitor of ABCB1 efflux.

33. The kit of claim 31, wherein the drug that reduces efflux is an inhibitor of ABCB1 ATPase activity.

34. The kit of claim 31, wherein the drug that reduces efflux is an anti-androgen drug.

35. The kit of claim 34, wherein the anti-androgen drug is bicalutamide, enzalutamide, or a combination thereof.

36. The kit of claim 35, wherein the anti-androgen drug is bicalutamide.

37. The kit of claim 35, wherein the anti-androgen drug is enzalutamide.

38. The kit of claim 31, wherein the taxane is docetaxel.

39. The kit of claim 31, wherein the container containing the taxane contains docetaxel and the container containing the drug that reduces efflux of the taxane from the cell contains bicalutamide, enzalutamide, or a combination thereof.

40. The kit of claim 31, wherein the kit further comprises a label with instructions for administering the taxane and the drug that reduces efflux of the taxane from the cell.
